(19) United States
(12) Patent Application Publication
(10) Pub. No.: US 2026/0353049 A1
(43) Pub. Date: Nov. 5, 2026

(54) REDUNDANT MULTI-SENSOR DETECTION SYSTEM, CARTRIDGE AND SYSTEM FOR ANALYTE IDENTIFICATION

(71) Applicants: MATTHEW HUMMER, SHAKER HEIGHTS, OH (US); GREGORY J. HUMMER, SHAKER HEIGHTS, OH (US)

(72) Inventors: MATTHEW HUMMER, SHAKER HEIGHTS, OH (US); GREGORY J. HUMMER, SHAKER HEIGHTS, OH (US)

(21) Appl. No.: 19/711,025

(22) Filed: Jun. 17, 2026

Related U.S. Application Data
(63) Continuation-in-part of application No. 19/647,860, filed on Apr. 14, 2026.
Application No. 19/647,860 is a continuation of application No. 19/260,530, filed on Jul. 6, 2025, now Pat. No. 12,600,636.
Application No. 19/260,530 is a continuation-in-part of application No. 18/928,124, filed on Oct. 27, 2024, now abandoned.
Application No. 18/928,124 is a continuation-in-part of application No. 18/896,643, filed on Sep. 25, 2024.
Application No. 18/896,643 is a continuation of application No. 18/440,925, filed on Feb. 13, 2024, now abandoned.
Application No. 18/440,925 is a continuation of application No. 17/505,611, filed on Oct. 19, 2021, now Pat. No. 11,896,372.
Application No. 17/505,611 is a continuation-in-part of application No. 17/324,085, filed on May 18, 2021, now Pat. No. 11,340,210.
Application No. 17/324,085 is a continuation-in-part of application No. 16/926,702, filed on Jul. 11, 2020, now Pat. No. 11,172,339.
Application No. 16/926,702 is a continuation-in-part of application No. 16/926,701, filed on Jul. 11, 2020, now Pat. No. 11,179,061.

(51) Int. Cl. H04W 4/38; B64U 10/13; B64U 101/19; B64U 101/55; C01B 32/194; C09D 11/037; C12Q 1/04; C12Q 1/70; G01N 33/483; G01N 33/50; G01S 19/13; G01S 19/39; G08B 21/12; H04Q 9/00

(52) U.S. Cl. CPC H04W 4/38; C01B 32/194; C09D 11/037; C12Q 1/04; C12Q 1/70; G01N 33/483; G01N 33/50; G01S 19/13; G01S 19/39; G08B 21/12; H04Q 9/00; B64U 10/13; B64U 2101/19; B64U 2101/55; B64U 2201/104; B64U 2201/20; C01B 2204/32; C01P 2002/82; C01P 2006/10; C01P 2006/12; C01P 2006/22; H04Q 2209/40; H04Q 2209/50

(57) ABSTRACT

A system, cartridge assembly, and method for detecting an analyte using a plurality of sensors arranged in an array are disclosed. A cartridge includes multiple sensing regions configured to receive portions of a sample, and an instrument is configured to receive the cartridge and acquire electrical signals over time from the plurality of sensors. One or more processors receive the electrical signals, preprocess the electrical signals, classify the electrical signals based on changes in one or more electrical characteristics over time, and aggregate classifications or signal characteristics from a subset of sensors associated with the same analyte target. The processors determine the presence or absence of the analyte based on aggregated sensor behavior from the subset of sensors rather than based exclusively on an output from any individual sensor.

### CROSS-REFERENCE TO RELATED APPLICATIONS

This Patent Application is a Continuation-in-part and claims priority to U.S. patent application Ser. No. 19/647,860 , filed on Apr. 14, 2026, entitled “FUNCTIONALIZED GRAPHENE AND METHODS OF USE THEREOF”, by Matthew Hummer et al., which is a Continuation of U.S. Pat. No. 12,600,636 , filed on Jul. 6, 2025, entitled “FUNCTIONALIZED GRAPHENE AND METHODS OF USE THEREOF”, by Matthew Hummer et al., which is a Continuation-in-part of U.S. patent application Ser. No. 18/928,124 , filed on Oct. 27, 2024, entitled “GRAPHENE INK COMPOSITION AND METHODS OF USE THEREOF”, by Matthew Hummer et al., which is a Continuation-in-part of U.S. patent application Ser. No. 18/896,643, filed on Sep. 25, 2024, entitled “DEVICE FOR READING, PROCESSING AND TRANSMITTING TEST RESULT DATA FOR PATHOGENS OR VIRUSES IN FLUID TEST SAMPLES”, which is a Continuation of U.S. patent application Ser. No. 18/440,925, filed on Feb. 13, 2024, entitled “DEVICE FOR READING, PROCESSING AND TRANSMITTING TEST RESULT DATA FOR PATHOGENS OR VIRUSES IN FLUID TEST SAMPLES”, by Matthew Hummer et al., which is a Continuation of U.S. patent application Ser. No. 17/505,611, filed on Oct. 19, 2021, entitled “DEVICE FOR READING, PROCESSING AND TRANSMITTING TEST RESULT DATA FOR PATHOGENS OR VIRUSES IN FLUID TEST SAMPLES”, by Matthew Hummer et al., which is a Continuation-in-part of U.S. patent application Ser. No. 17/324,085 , filed on May 18, 2021, entitled “METHOD AND DEVICES FOR DETECTING VIRUSES AND BACTERIAL PATHOGENS”, by Matthew Hummer et al., which is a Continuation-in-part of U.S. patent application Ser. No. 16/926,702 , filed on Jul. 11, 2020, entitled “METHOD AND DEVICES FOR DETECTING CHEMICAL COMPOSITIONS AND BIOLOGICAL PATHOGENS”, by Gregory J. Hummer et al. and a Continuation-in-part of U.S. patent application Ser. No. 16/926,701 , filed on Jul. 11, 2020, entitled “METHOD AND DEVICES FOR DETECTING VIRUSES AND BACTERIAL PATHOGENS”, by Gregory J. Hummer et al., all which are incorporated herein by reference.

### BACKGROUND OF THE INVENTION

Detection systems for identifying biological analytes, nucleic acids, oligonucleotides, DNA, RNA, cell-free nucleic acids, proteins, peptides, antibodies, antigens, aptamers, pathogens, biomarkers, metabolites, chemical compounds, environmental contaminants, toxins, mycotoxins, allergens, gases, volatile compounds, microorganisms, viruses, bacteria, fungi, parasites, cells, extracellular vesicles, and other target substances are used in a wide variety of medical, clinical, diagnostic, molecular testing, research, pharmaceutical, biotechnology, veterinary, agricultural, food safety, environmental monitoring, biodefense, industrial process control, occupational safety, forensic, and public health applications. Such systems may be used for screening, diagnosis, monitoring, surveillance, quality control, therapeutic selection, treatment response assessment, contamination detection, and other analytical or decision-making purposes across centralized laboratories, decentralized testing environments, point-of-care settings, field-deployable systems, and automated instrumentation platforms. Existing systems may utilize cartridges, assay chambers, microfluidic structures, biosensors, electrochemical sensors, optical sensors, impedance sensors, current-based sensors, or other sensing architectures configured to evaluate a sample and generate one or more measurable outputs. Such systems may further include reader instruments, portable diagnostic platforms, cloud-connected processing systems, software applications, and data analysis engines configured to acquire, process, transmit, and display detection results. In many implementations, a sample may be distributed across multiple sensing regions or wells to permit parallel testing, redundancy, multiplexing, calibration, control analysis, or comparative evaluation of signals generated from the sample.

Conventional detection platforms may further utilize signal processing algorithms, pattern recognition systems, statistical analysis techniques, machine learning models, thresholding approaches, confidence scoring mechanisms, and other computational methods to analyze sensor outputs and generate determinations relating to the presence, absence, concentration, classification, or characterization of one or more analytes. Such systems may include portable or benchtop readers configured to communicate with remote servers, mobile applications, cloud computing systems, databases, reporting interfaces, or external computing devices. Existing products and systems may also include disposable cartridges, sensor arrays, reagent structures, fluid distribution layers, conductive routing layers, and various electronic or software-based architectures configured to facilitate automated or semi-automated testing workflows and result generation.

### SUMMARY OF THE INVENTION

The present invention relates generally to systems, devices, cartridges, instruments, algorithms, software platforms, and methods for detecting, identifying, classifying, quantifying, monitoring, or otherwise evaluating one or more analytes within a sample using a plurality of sensors arranged in an array. In certain embodiments, the invention includes a cartridge comprising a plurality of sensors configured to receive or interact with a sample, an instrument configured to receive the cartridge and acquire electrical signals from the plurality of sensors, and one or more processors configured to process the electrical signals and determine the presence or absence of one or more target analytes. The processors may classify electrical signals based on changes in electrical current over time, including direction, magnitude, rate of change, slope, waveform characteristics, temporal response characteristics, impedance characteristics, conductivity characteristics, electrochemical behavior, and other signal parameters. In certain embodiments, the determination of analyte presence or absence is based on signals, classifications, weighted classifications, aggregated responses, confidence scores, statistical relationships, or combinations thereof generated from a subset of sensors assigned to a particular analyte. In certain embodiments, analyte determination is performed based on aggregated classifications, aggregated signal characteristics, aggregated feature vectors, aggregated waveform characteristics, or combinations thereof generated from multiple sensors associated with the same analyte target rather than based exclusively on an output from any individual sensor.

In certain embodiments, the invention utilizes a redundancy-based sensor architecture in which multiple sensors are assigned to a common analyte target. Rather than relying exclusively on the output of a single sensing element, the system may evaluate responses from multiple sensors associated with the analyte and determine the presence or absence of the analyte based on combined or aggregated sensor behavior. Such aggregation may improve robustness, reproducibility, fault tolerance, sensitivity, specificity, selectivity, signal reliability, and resistance to sensor variability. In certain embodiments, the processor is configured to determine the presence of an analyte based on one or more signals generated by at least one sensor within the subset and/or based on aggregated responses from a plurality of sensors within the subset. The system may therefore permit analyte detection even where individual sensor responses are weak, partially degraded, noisy, inconsistent, or otherwise non-ideal. In certain embodiments, analyte determination utilizes combined evaluation of multiple sensors even when one or more sensors independently exhibit strong positive behavior.

In certain embodiments, the processor applies one or more classification algorithms configured to classify electrical signals into multiple categories. By way of example and without limitation, classifications may include strong positive, weak positive, strong negative, weak negative, invalid, inconclusive, indeterminate, noisy, unstable, drifting, saturated, or control-failure classifications. In certain embodiments, a strong positive classification may correspond to an increasing electrical current over time, including stepwise transitions, exponential increases, stabilized elevated responses, or other signal behaviors associated with target binding or analyte interaction. In certain embodiments, classification is based on one or more electrical characteristics, including current, voltage, impedance, capacitance, resistance, conductivity, electrochemical characteristics, frequency-domain characteristics, waveform characteristics, phase characteristics, transient characteristics, signal stability characteristics, binding or hybridization characteristics, kinetic characteristics, ionic strength measurements, ionic concentration measurements, ionic activity measurements, pH measurements or combinations thereof, evaluated over time, across multiple sensors, or relative to one or more baseline, reference, calibration, or threshold conditions.

A strong negative classification may correspond to decreasing current behavior, suppression characteristics, absence of binding, inverse response characteristics, or other negative detection profiles. Weak positive classifications may include lower magnitude responses, delayed responses, partial responses, non-monotonic behavior, oscillatory behavior, or mixed positive and negative segments over time. Invalid classifications may include flat responses, untriggered sensors, unstable signals, disconnected sensors, saturated responses, electrically noisy signals, or sensor malfunctions.

In certain embodiments, one or more processors are configured to apply a confidence scoring function to aggregated sensor responses. The confidence scoring function may be based on one or more parameters, including the number of sensors classified as positive, the magnitude of electrical signals, consistency across sensors, signal-to-noise ratios, response timing, waveform similarity, temporal alignment, sensor weighting factors, calibration data, environmental conditions, historical sensor behavior, control sensor outputs, or machine learning-derived parameters. Confidence scores may be represented numerically, statistically, probabilistically, graphically, optically, colorimetrically, textually, electronically, algorithmically, digitally, visually, audibly, or through other representations. In certain embodiments, analyte presence may be determined when one or more strong positive classifications are detected or when multiple weak positive classifications collectively exceed a confidence threshold. In certain embodiments, strong negative classifications may override weak positive classifications. In other embodiments, weighted balancing, Bayesian analysis, probabilistic models, neural networks, ensemble classifiers, support vector machines, regression models, or other computational frameworks may be utilized. In certain embodiments, confidence scoring may further incorporate aggregated feature vectors, temporal agreement between sensing regions, waveform similarity metrics, derivative similarity metrics, spectral similarity metrics, or machine learning-derived weighting parameters.

In certain embodiments, the system may utilize machine learning or artificial intelligence models trained using historical sensor data, labeled datasets, calibration datasets, control datasets, population datasets, or continuously updated cloud datasets. Such models may be configured to identify patterns associated with analyte presence, sensor drift, environmental variability, assay degradation, cartridge variability, or user-specific conditions. In certain embodiments, the machine learning systems may continuously improve through additional uploaded data obtained from distributed instruments operating across multiple geographic locations. The processing architecture may therefore permit adaptive thresholding, dynamic calibration, individualized baselines, predictive analytics, anomaly detection, and continuously evolving classification criteria.

In certain embodiments, the cartridge comprises a layered structure including one or more sample interface layers, fluid distribution layers, sensor sheets, conductive routing layers, adhesive layers, membranes, reservoirs, absorbent structures, venting structures, sealing structures, reagent regions, microfluidic channels, or support substrates. The cartridge may include one or more sample receiving regions configured to receive blood, plasma, serum, saliva, urine, sputum, mucus, cerebrospinal fluid, synovial fluid, lymphatic fluid, interstitial fluid, pleural fluid, peritoneal fluid, bronchoalveolar lavage fluid, fecal samples, stool samples, semen, vaginal fluids, cervical samples, nasopharyngeal samples, swab extracts, tissue extracts, biopsy samples, cell lysates, cultured cells, microbial cultures, environmental samples, air samples, water samples, soil samples, industrial samples, food samples, agricultural samples, chemical samples, biological fluids, purified analyte preparations, processed sample materials, diluted sample materials, extracted nucleic acid samples, or other sample materials. In certain embodiments, the sample may be distributed across multiple sensors, wells, sensing chambers, or detection regions to facilitate parallel analysis and redundant detection.

In certain preferred embodiments, the sensor sheet and associated conductive routing structures are fabricated using screen printing processes in which conductive inks are deposited onto a substrate to form sensing regions, conductive traces, electrodes, routing pathways, contact pads, or other electrically conductive structures associated with the sensing system. The screen-printing processes may utilize conductive inks comprising conductive materials, carbon materials, allotrope of carbon materials, graphite materials, graphene materials, graphene oxide materials, reduced graphene oxide materials, carbon black materials, fullerene materials, MXene materials, van der Waals materials, two-dimensional materials, layered materials, silver materials, gold materials, copper materials, platinum materials, palladium materials, nickel materials, aluminum materials, conductive polymers, metallic nanoparticles, metal oxides, nanotube materials, carbon nanotube materials, nanowire materials, composite conductive materials, semiconductive materials, hybrid conductive materials, functionalized conductive materials, nanomaterial-based conductive materials or combinations thereof. Screen printing fabrication may permit scalable, cost-effective, high-volume manufacturing of disposable or semi-disposable sensor cartridges while maintaining suitable electrical performance for analyte detection operations.

In certain preferred embodiments, the sensor sheet comprises a screen-printed circuit structure formed on a flexible or rigid substrate using one or more conductive inks deposited through patterned screens. The screen-printed circuit structure may include conductive traces, sensing electrodes, reference electrodes, counter electrodes, interconnect pathways, shielding regions, multiplexed routing structures, or connector interfaces configured to electrically couple the sensing regions to one or more external processing systems or reader instruments.

Alternative embodiments may utilize printed circuit board fabrication processes, semiconductor fabrication processes, thin-film deposition processes, sputtering processes, vapor deposition processes, chemical vapor deposition processes, physical vapor deposition processes, atomic layer deposition processes, electroplating processes, electroless plating processes, electroless nickel immersion gold, etching processes, gold fired inks, metallic overglazes, photolithographic processes, lift-off lithography, electron beam lithography, extreme ultraviolet lithography, stone lithography, offset lithography, transfer lithography, inkjet printing processes, aerosol deposition processes, screen-printing processes, gravure printing processes, flexographic printing processes, stencil printing processes, laser patterning processes, flexible circuit fabrication processes, additive manufacturing processes, microfabrication processes, nanofabrication processes, roll-to-roll manufacturing processes, lamination processes, transfer printing processes, or combinations thereof. Such alternative fabrication approaches may be utilized alone or in combination with screen printing fabrication processes, depending on desired manufacturing characteristics, sensing architectures, analyte targets, electrical performance requirements, or commercial implementation considerations.

In certain embodiments, the sensor sheet comprises a plurality of individually addressable sensors arranged in rows, columns, grids, matrices, concentric patterns, staggered arrays, or other geometric arrangements. Each sensor may include one or more active sensing regions, electrodes, conductive pathways, interconnect regions, contact pads, nanostructures, coatings, membranes, binding agents, probes, capture molecules, proteins, antibodies, aptamers, oligonucleotides, nucleic acid probes, molecularly imprinted polymers, graphene structures, nanotubes, nanowires, metallic structures, semiconductor structures, MXene structures, van der Waals material structures, two-dimensional material structures, conductive polymer structures, metal oxide structures, composite material structures, functionalized nanomaterial structures, catalytic structures, dielectric structures, passivation layers, insulating layers, biofunctional layers, receptor molecules, ligands, enzymes, peptides, linker molecules, self-assembled monolayers, or other sensing elements. The sensors may be configured to detect nucleic acids, mutations, pathogens, proteins, biomarkers, antibodies, antigens, toxins, metabolites, chemicals, pharmaceutical compounds, contaminants, cells, microorganisms, viruses, bacteria, fungi, parasites, genetic variants, polymorphisms, peptides, enzymes, hormones, cytokines, allergens, environmental agents, industrial agents, volatile compounds, gases, heavy metals, pollutants, therapeutic agents, drug compounds, disease markers, cancer markers, infectious disease markers, inflammatory markers, cardiac markers, neurological markers, or other target substances.

In certain embodiments, the instrument comprises a portable or benchtop reader configured to electrically interface with the cartridge. The instrument may include one or more processors, analog front-end circuitry, amplifiers, multiplexers, analog-to-digital converters, communication modules, displays, user interfaces, wireless transceivers, memory devices, batteries, charging systems, cloud communication systems, or embedded software systems. The instrument may apply electrical bias conditions, excitation signals, current measurements, voltage measurements, impedance measurements, frequency sweeps, pulse sequences, or other interrogation techniques to the sensors. In certain embodiments, the instrument acquires sensor data over time and transmits the data to a local processor, remote server, cloud computing platform, mobile application, or distributed processing network for analysis.

In certain embodiments, the invention further includes cloud-based processing systems configured to receive sensor data from one or more instruments and perform remote classification, aggregation, confidence scoring, storage, analytics, reporting, monitoring, software updating, calibration updating, or machine learning operations. The cloud system may communicate with mobile applications, smartphones, tablets, laptops, hospital systems, data conversion software, laboratory systems, electronic medical record systems, physician portals, patient portals, regulatory databases, epidemiological monitoring systems, or third-party computing and data management systems. Results may be displayed locally on the instrument, on a mobile device, through a cloud portal, through downloadable reports, or through integrated healthcare information systems.

In certain embodiments, the invention further includes workflows for performing analyte detection assays. Such workflows may include sample preparation, plasma preparation, lysis, filtration, purification, reagent mixing, incubation, denaturation, amplification, dilution, deposition onto sensor arrays, sensor interrogation, signal acquisition, signal classification, algorithmic classification, aggregation analysis, confidence scoring, cloud transmission, electronic transmission, automated interpretation, and result output. In certain embodiments, a sample may be loaded using pipettes, automated liquid handlers, robotic systems, capillary systems, syringes, cartridges, reservoirs, channels, pumps, valves, or microfluidic transport structures. Samples may be deposited into multiple wells or sensing regions in parallel to facilitate simultaneous redundant measurements.

In certain embodiments, the system may generate results within seconds or minutes following sample loading. Results may include positive or negative determinations, mutation detection determinations, pathogen identification, concentration measurements, confidence levels, trend analyses, quantitative outputs, graphs, waveform visualizations, historical comparisons, alerts, recommendations, classifications, diagnostic determinations, monitoring outputs, predictive outputs, probability scores, risk assessments, notifications, reports, dashboards, or other outputs. The system may further include encryption systems, secure cloud transmission systems, audit logging systems, authentication systems, user management systems, cartridge registration systems, QR code systems, calibration tracking systems, quality control systems, data storage systems, software update systems, remote monitoring systems, algorithm training systems, machine learning systems, wireless communication systems, or compliance systems.

In certain embodiments, the invention may be utilized for medical diagnostics, companion diagnostics, infectious disease detection, oncology testing, mutation analysis, liquid biopsy testing, pharmacogenomics, environmental monitoring, food safety testing, biodefense applications, veterinary diagnostics, industrial process monitoring, agricultural testing, toxicology testing, research applications, laboratory workflows, decentralized testing, point-of-care testing, at-home testing, field testing, screening applications, surveillance applications, therapeutic monitoring, treatment selection, disease monitoring, prognostic testing, biomarker analysis, genomic testing, proteomic testing, precision medicine applications, public health applications, emergency response applications, wearable sensing applications, continuous monitoring applications, or other detection applications. The disclosed systems and methods may be implemented in centralized laboratory environments, distributed testing networks, portable diagnostic systems, mobile testing systems, cloud-connected testing ecosystems, or combinations thereof.

The foregoing summary is provided to introduce a selection of concepts in simplified form that are further described below in the Detailed Description. This summary is not intended to identify all claimed subject matter or to limit the scope of the claimed subject matter in any manner.

### BRIEF DESCRIPTION OF THE DRAWINGS

FIG. 1 illustrates a detection system including an instrument configured to receive a cartridge containing a plurality of sensors and acquire electrical signals associated with the detection of one or more analytes within a sample.

FIG. 2 illustrates an exploded view of the instrument showing internal electronic components, processing circuitry, connector structures, and housing portions configured to support acquisition and processing of electrical sensor signals.

FIG. 3 illustrates a cartridge configured for insertion into the instrument and includes a plurality of sensing regions arranged to receive a sample and generate electrical responses associated with analyte detection.

FIG. 4 illustrates an exploded view of the cartridge showing multiple layers, including sample receiving structures, fluid distribution regions, sensor layers, conductive routing structures, and support components configured to facilitate analyte detection.

FIG. 5 illustrates a sensor layer comprising a plurality of sensor elements arranged in an array and electrically coupled through conductive routing structures for the acquisition of electrical signals from multiple sensing regions.

FIG. 6A illustrates representative signal generation and signal behavior associated with detection of an analyte.

FIG. 6B illustrates representative examples of positive electrical response characteristics evaluated over time.

FIG. 6C illustrates representative examples of negative electrical response characteristics evaluated over time.

FIG. 7 illustrates a system and method for processing electrical signals, classifying sensor responses, aggregating classifications across multiple sensors, and determining the presence or absence of an analyte.

FIG. 8 illustrates a workflow for detecting an analyte using a multi-sensor system, including sample preparation, sample loading, sensor interrogation, signal processing, and result generation operations.

FIG. 9 illustrates a workflow for operating a detection system and delivering results to a user, including cartridge registration, test initiation, data transmission, result processing, and result output operations.

### DETAILED DESCRIPTION OF THE INVENTION

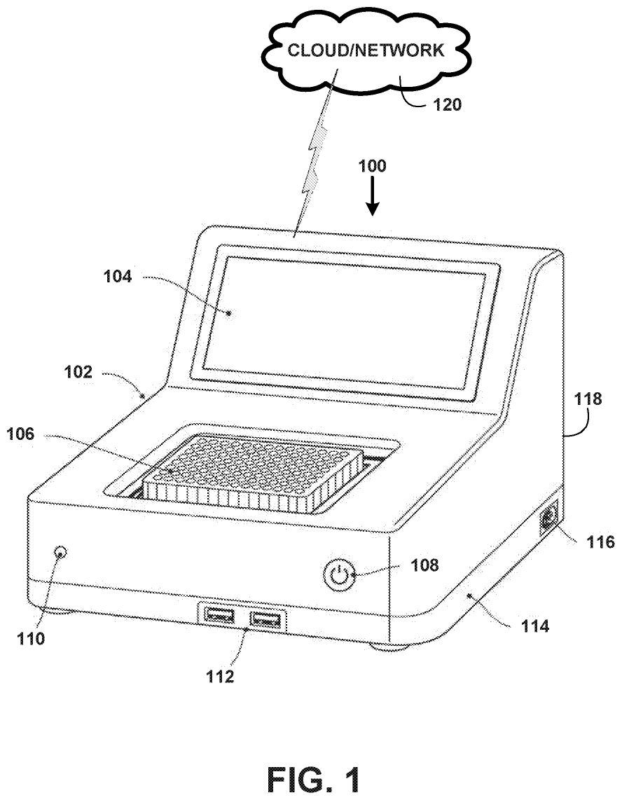

FIG. 1 shows an exemplary detection system 100 that includes an instrument or reader device 102 having a display screen 104 and configured to receive a cartridge 106, such as a multi-sensor cartridge, for performing analyte detection operations associated with one or more samples. The display screen 104 is configured to present operational information, assay instructions, cartridge status information, analyte detection outputs, confidence scores, connectivity indicators, calibration prompts, graphical user interfaces, or other operational information associated with operation of the detection system 100. The display screen 104 may comprise a liquid crystal display LCD, thin-film transistor TFT display, organic light-emitting diode OLED display, electrophoretic display, segmented display, touchscreen display, or other visual output device. Certain embodiments may additionally include audible indicators, haptic feedback systems, vibration systems, voice output systems, indicator lights, or external display systems configured to supplement operation of the display screen 104.

In certain embodiments, the cartridge 106 comprises a disposable, semi-disposable, reusable, refillable, or hybrid multi-sensor cartridge structure configured to receive a sample and support analyte detection operations. The cartridge 106 may include one or more polymeric bodies, laminated structures, molded structures, microfluidic layers, sensor sheets, conductive routing layers, adhesive layers, membranes, reservoirs, vent structures, sample receiving regions, sealing structures, reagent storage regions, absorbent structures, or fluid distribution structures. The cartridge 106 may further include rigid, semi-rigid, or flexible structures fabricated from polycarbonate, cyclic olefin copolymer COC, cyclic olefin polymer COP, polyethylene terephthalate PET, polypropylene, silicone materials, glass substrates, ceramic substrates, printed circuit board materials, flexible circuit materials, or other suitable materials. In certain embodiments, the cartridge 106 includes a plurality of sensing regions configured to receive distributed portions of a sample. The sensing regions may include wells, chambers, capillary structures, porous substrates, conductive electrodes, nanostructured sensing surfaces, electrochemical sensing elements, impedance sensing structures, field effect transistor structures, optical sensing structures, or combinations thereof. In certain embodiments, subsets of sensing regions are associated with common analyte targets to facilitate redundant detection operations and aggregated signal analysis across multiple sensors assigned to the same analyte target.

The instrument 102 further includes a power button 108 configured to activate, deactivate, initialize, reset, or otherwise control the operation of the detection system 100. The power button 108 may comprise a mechanical push button, capacitive touch interface, membrane switch, dome switch, optical switch, pressure-sensitive interface, or other user-actuated control structure. In certain embodiments, the instrument 102 further includes a power light 110 configured to provide visual indications associated with the operation of the instrument 102. The power light 110 may indicate power availability, charging status, communication activity, operational readiness, cartridge detection, assay execution status, warning conditions, fault conditions, or low battery conditions. The power light 110 may comprise one or more light-emitting diodes LEDs, multicolor indicators, illuminated rings, segmented indicators, or other illumination structures configured to communicate operational states to a user.

In certain embodiments, the instrument 102 further includes one or more auxiliary ports 112 configured to support electrical communication, peripheral interfacing, charging operations, debugging operations, firmware updates, external sensor interfacing, or data transfer operations associated with the instrument 102. The auxiliary ports 112 may include USB interfaces, Ethernet interfaces, serial communication interfaces, SPI interfaces, I2C interfaces, CAN bus interfaces, HDMI interfaces, audio interfaces, memory card interfaces, or proprietary communication interfaces. Certain embodiments may additionally include wireless communication systems including Bluetooth communication modules, Wi Fi communication modules, cellular communication modules, NFC systems, RFID systems, Zigbee communication systems, LoRa communication systems, or combinations thereof, configured to communicate with mobile computing devices, cloud computing systems, laboratory systems, hospital systems, or external computing platforms.

The instrument 102 further includes a housing base 114 configured to structurally support the instrument 102 on a substantially planar surface during operation of the detection system 100. In certain embodiments, the housing base 114 includes elastomeric feet, vibration-damping structures, anti-slip materials, leveling structures, weighted stabilization members, mounting interfaces, or shock-absorbing structures configured to reduce movement during testing operations. Certain embodiments may further include inertial sensors, accelerometers, gyroscopes, tilt sensors, or motion-detection circuitry configured to identify movement of the instrument 102 during assay execution. In certain embodiments, testing operations may be suspended, recalibrated, flagged, or invalidated in response to detected movement exceeding one or more thresholds.

In certain embodiments, the instrument 102 further includes a power connection port 116 configured to receive electrical power from an external power source associated with the operation of the instrument 102. The power connection port 116 may interface with external power adapters, docking stations, charging assemblies, battery charging systems, USB power systems, or other external power infrastructures. The instrument 102 may further include one or more internal power systems configured to supply electrical power to analog front-end circuitry, processors, displays, communication systems, thermal control systems, or sensing systems associated with the detection system 100. Such power systems may include rechargeable batteries, lithium-ion batteries, lithium polymer batteries, nickel-metal hydride batteries, alkaline batteries, wireless charging systems, inductive charging systems, solar charging systems, or hybrid power architectures. In certain embodiments, the instrument 102 further includes power management circuitry configured to regulate voltages supplied to internal subsystems using voltage regulators, buck converters, boost converters, charging controllers, protection circuitry, battery management systems, current limiting circuitry, or thermal-monitoring circuitry.

The housing base 114 of the instrument 102 is configured to contain electrical, mechanical, thermal, computational, optical, and communication components associated with the operation of the detection system 100. In certain embodiments, the housing base 114 is part of a housing 118 and includes one or more polymeric materials, injection-molded structures, metallic frames, composite structures, thermally conductive structures, electromagnetic shielding structures, elastomeric sealing structures, or combinations thereof. The housing 118 may further include upper and lower enclosure portions coupled using fasteners, ultrasonic welding, adhesives, snap fit structures, threaded interfaces, magnetic couplings, living hinges, or other attachment mechanisms. In certain embodiments, the housing 118 includes internal support ribs, circuit board mounting structures, airflow channels, cartridge retention structures, alignment guides, heat dissipation structures, vibration isolation structures, or cable routing structures configured to support stable operation of the instrument 102.

In certain embodiments, the instrument 102 further includes one or more processors configured to control the operation of the detection system 100 and process acquired sensor data associated with the cartridge 106. The processors may include microprocessors, microcontrollers, digital signal processors DSPs, field programmable gate arrays, FPGAs, system on chip devices SoCs, graphics processing units GPUs, tensor processing units TPUs, neural network accelerators, application specific integrated circuits ASICs, programmable logic devices, or combinations thereof. In certain embodiments, one or more processors execute instructions stored within one or more memory systems to control cartridge interrogation, sensor addressing, signal acquisition, analog-to-digital conversion, filtering, feature extraction, classification, confidence scoring, cloud communication, user interface functions, or result generation operations.

The instrument 102 may further include one or more memory systems configured to store executable instructions, calibration information, assay definitions, sensor configuration information, temporary buffers, encryption keys, historical test data, machine learning models, firmware images, operating system instructions, audit logs, user profiles, or cloud synchronization data. The memory systems may include volatile memory, nonvolatile memory, flash memory, EEPROM devices, RAM, DRAM, SDRAM, MRAM, FRAM, solid-state storage devices, removable storage media, or cloud-linked storage architectures.

In certain embodiments, the instrument 102 includes analog front-end circuitry configured to interface with sensing regions contained within the cartridge 106. The analog front end circuitry may include transimpedance amplifiers, instrumentation amplifiers, operational amplifiers, current-sensing circuitry, voltage-sensing circuitry, multiplexers, filters, impedance measurement circuitry, excitation signal generators, waveform generators, analog switches, reference voltage circuitry, analog-to-digital converters, digital-to-analog converters, low-noise amplifiers, shielding structures, grounding systems, or signal conditioning circuitry. In certain embodiments, the analog front-end circuitry is configured to acquire current versus time data associated with interactions occurring at sensing regions contained within the cartridge 106.

In certain embodiments, the instrument 102 and the cartridge 106 operate together to perform redundant analyte detection using multiple sensing regions associated with a common analyte target. One or more processors may aggregate responses across subsets of sensing regions and determine analyte presence or absence based on combined signal information rather than reliance upon a single sensing region response. Such aggregation operations may include weighted voting operations, confidence scoring operations, probabilistic inference operations, statistical averaging operations, threshold analysis operations, machine learning classification operations, ensemble model operations, Bayesian fusion operations, or temporal aggregation operations.

In certain embodiments, the detection system 100 further includes a cloud network 120 configured to communicate with the instrument 102 and perform remote processing, storage, analytics, synchronization, or management operations associated with analyte detection activities. The cloud network 120 may include one or more cloud computing platforms, distributed computing systems, remote servers, edge computing systems, virtualized computing environments, containerized processing systems, database clusters, or network management infrastructures configured to process data acquired from the cartridge 106. In certain embodiments, sensor data, classification outputs, confidence scores, calibration information, firmware updates, machine learning models, user profiles, assay definitions, audit records, or operational diagnostics are transmitted between the instrument 102 and the cloud network 120 through encrypted communication channels. The cloud network 120 may further support remote machine learning classification operations, epidemiological monitoring, mutation analysis, distributed calibration management, population-level analytics, fleet management operations, predictive maintenance operations, user authentication operations, laboratory information system integration, electronic medical record integration, or remote reporting operations associated with the detection system 100.

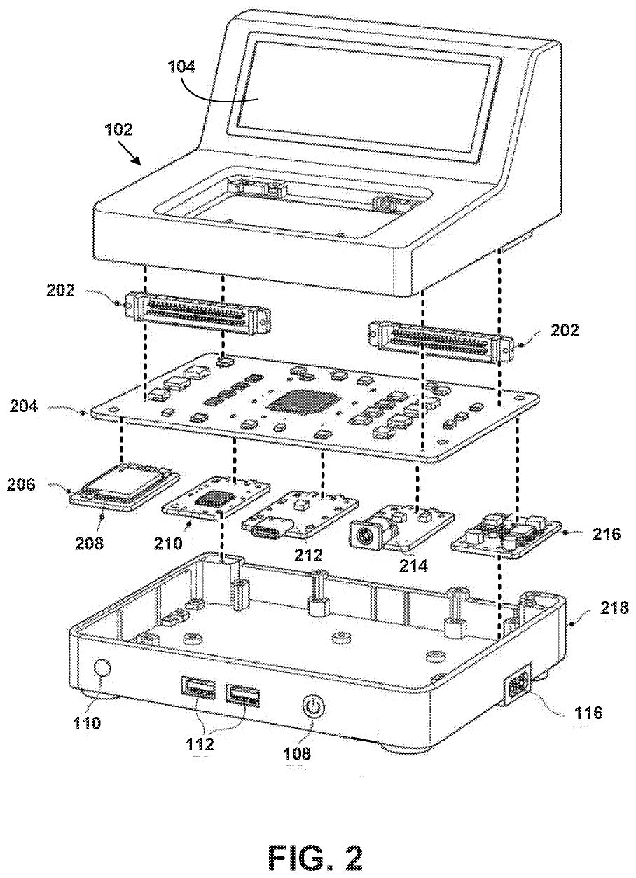

With reference to FIG. 2, the instrument 102 includes a housing 218 configured to contain electrical, mechanical, thermal, computational, optical, and communication components associated with operation of the detection system 100 of FIG. 1. In certain embodiments, the housing 218 includes one or more polymeric materials, injection molded structures, metallic frames, composite structures, thermally conductive structures, electromagnetic shielding structures, elastomeric sealing structures, or combinations thereof. The housing 218 may include upper and lower enclosure portions coupled using fasteners, ultrasonic welding, adhesives, snap fit structures, threaded interfaces, magnetic couplings, living hinges, or other attachment mechanisms. In certain embodiments, the housing 218 includes internal support ribs, circuit board mounting structures, alignment guides, airflow channels, heat dissipation structures, vibration isolation structures, or cable routing structures configured to support stable operation of the instrument 102.

The instrument 102 further includes a display screen 104 configured to present operational information associated with the detection system 100 of FIG. 1. In certain embodiments, the display screen 104 presents assay instructions, cartridge status information, analyte detection outputs, confidence scores, calibration prompts, connectivity indicators, error notifications, graphical user interfaces, system diagnostics, or other operational information associated with operation of the instrument 102. The display screen 104 may comprise a liquid crystal display LCD, thin-film transistor TFT display, organic light-emitting diode OLED display, electrophoretic display, segmented display, touchscreen display, or other visual output device. Certain embodiments may additionally include audible indicators, haptic feedback systems, vibration systems, voice output systems, external displays, or indicator lights configured to supplement operation of the display screen 104.

In certain embodiments, the instrument 102 includes electrical connectors 202 configured to electrically interface with the cartridge 106 of FIG. 1. The electrical connectors 202 may include pogo pins, spring contacts, conductive elastomers, zero insertion force connectors, card edge connectors, flex circuit connectors, conductive pads, pressure contacts, anisotropic conductive films, or other conductive interconnect structures configured to electrically couple sensing regions within the cartridge 106 of FIG. 1 to circuitry contained within the instrument 102. In certain embodiments, the electrical connectors 202 are positioned within a cartridge receiving region of the housing 218 and are configured to align with conductive contact regions associated with the cartridge 106 during insertion of the cartridge 106 of FIG. 1 into the instrument 102.

The instrument 102 further includes a main circuit board 204 configured to support electrical communication, signal processing, power distribution, and computational operations associated with the detection system 100 of FIG. 1. In certain embodiments, the main circuit board 204 comprises a printed circuit board PCB including conductive routing structures, multilayer interconnect structures, grounding regions, shielding regions, power distribution pathways, connector interfaces, memory interfaces, processor interfaces, analog front-end circuitry, wireless communication circuitry, or combinations thereof. The main circuit board 204 may be fabricated using rigid PCB materials, flexible PCB materials, rigid flex PCB structures, ceramic circuit substrates, insulated metal substrates, or other electronic substrate structures suitable for supporting operation of the instrument 102.

In certain embodiments, the instrument 102 includes a plurality of electronic chips and circuits 206 associated with the operation of the power light 110. The plurality of electronic chips and circuits 206 may include light control circuitry, driver circuitry, voltage regulation circuitry, illumination control systems, pulse width modulation systems, current limiting circuitry, indicator control logic, or other electronic systems configured to control visual operational indicators associated with the power light 110. The power light 110 may indicate power availability, charging status, communication activity, operational readiness, cartridge detection, assay execution status, warning conditions, fault conditions, or low battery conditions associated with operation of the instrument 102.

The instrument 102 further includes a plurality of electronic chips and circuits 208, including one or more microchips configured to control processing operations associated with the detection system 100 of FIG. 1. The plurality of electronic chips and circuits 208 may include microprocessors, microcontrollers, digital signal processors DSPs, field programmable gate arrays FPGAs, system on chip devices SoCs, graphics processing units GPUs, tensor processing units TPUs, neural network accelerators, memory controllers, communication controllers, programmable logic devices, application specific integrated circuits ASICs, or combinations thereof. In certain embodiments, the plurality of electronic chips and circuits 208 execute instructions configured to control cartridge interrogation, sensor addressing, analog-to-digital conversion, signal acquisition, filtering, feature extraction, confidence scoring, cloud communication, user interface operations, or analyte classification operations associated with the detection system 100 of FIG. 1.

In certain embodiments, the instrument 102 further includes a plurality of electronic chips and circuits 210 associated with the auxiliary ports 112. The plurality of electronic chips and circuits 210 may include USB controller circuitry, Ethernet controller circuitry, serial communication circuitry, SPI circuitry, I2C circuitry, CAN bus circuitry, HDMI circuitry, audio circuitry, wireless communication circuitry, or combinations thereof configured to support data transfer, firmware updates, charging operations, debugging operations, peripheral interfacing, or communication with external computing systems. In certain embodiments, the auxiliary ports 112 communicate with mobile computing devices, laboratory systems, cloud computing systems, hospital systems, or external computing platforms through wired or wireless communication channels.

The instrument 102 further includes a plurality of electronic chips and circuits 212 associated with the operation of the power button 108. The plurality of electronic chips and circuits 212 may include switch monitoring circuitry, debounce circuitry, low power wake circuitry, interrupt generation circuitry, capacitive sensing circuitry, voltage monitoring circuitry, or operational mode control circuitry configured to control activation, deactivation, initialization, reset operations, standby operations, or calibration operations associated with the instrument 102.

In certain embodiments, the instrument 102 includes a plurality of electronic chips and circuits 214 associated with the operation of the power connection port 116. The plurality of electronic chips and circuits 214 may include voltage regulators, buck converters, boost converters, charging controllers, battery management systems, protection circuitry, current limiting circuitry, thermal monitoring circuitry, AC to DC conversion circuitry, filtering circuitry, or power conditioning circuitry configured to regulate electrical power supplied to components contained within the instrument 102. In certain embodiments, the power connection port 116 is configured to interface with external power adapters, charging assemblies, docking stations, USB power systems, or other external power infrastructures associated with the operation of the detection system 100 of FIG. 1.

The instrument 102 further includes a lower housing 216 configured to support and enclose internal electronic components associated with the operation of the detection system 100 of FIG. 1. In certain embodiments, the lower housing 216 includes structural mounting features, thermal dissipation structures, vibration isolation structures, airflow channels, fastening structures, connector alignment structures, or support features configured to mechanically support the main circuit board 204 and the plurality of electronic chips and circuits 206, 208, 210, 212, 214 and 216. The lower housing 218 may comprise polymeric materials, composite materials, metallic materials, thermally conductive materials, or combinations thereof configured to provide structural integrity and environmental protection associated with the operation of the instrument 102.

In certain embodiments, the instrument 102 communicates with a cloud network 120 of FIG. 1 configured to support remote processing, analytics, synchronization, management, or storage operations associated with the detection system 100 of FIG. 1. The cloud network 120 of FIG. 1 may include one or more cloud computing platforms, distributed computing systems, remote servers, edge computing systems, virtualized computing environments, database systems, or network management infrastructures configured to process operational data associated with the instrument 102 and the cartridge 106 of FIG. 1. In certain embodiments, the cloud network 120 supports machine learning classification operations, epidemiological monitoring operations, calibration management operations, remote reporting operations, user authentication operations, fleet management operations, predictive maintenance operations, laboratory information system integration, or electronic medical record integration associated with the detection system 100 of FIG. 1.

With reference to FIG. 2, the cartridge 106 of FIG. 1 of the system 100 of FIG. 1 includes a multilayer sensing structure configured to receive a sample, distribute the sample to a plurality of sensing regions, electrically interface with the instrument 102, and facilitate acquisition of electrical data associated with one or more analytes. In certain embodiments, FIG. 2 illustrates electrical connectors 202, a main circuit board 204, a plurality of electronic chips and circuits 206 associated with operation of the power light 110, a plurality of electronic chips and circuits 208 including one or more microchips configured to control signal acquisition and processing operations, a plurality of electronic chips and circuits 210 associated with the auxiliary ports 112, a plurality of electronic chips and circuits 212 associated with operation of the power button 108, a plurality of electronic chips and circuits 214 associated with operation of the power connection port 116, and a lower housing 216 configured to support and enclose internal electronic components associated with operation of the instrument 102. The various structures illustrated in FIG. 2 may be assembled into an integrated electronic and mechanical architecture configured to support operation of the system 100 of FIG. 1.

In certain embodiments, the electrical connectors 202 are configured to electrically interface with the cartridge 106 of FIG. 1 and communicate electrical signals between sensing structures contained within the cartridge 106 of FIG. 1 and circuitry contained within the instrument 102. The electrical connectors 202 may include pogo pins, spring contacts, conductive elastomers, zero insertion force connectors, card edge connectors, flex circuit connectors, conductive pads, pressure contacts, anisotropic conductive films, or other conductive interconnect structures configured to electrically couple sensing regions within the cartridge 106 of FIG. 1 to signal acquisition circuitry associated with the instrument 102. In certain embodiments, the electrical connectors 202 are positioned within a cartridge receiving region of the housing 218 and are configured to align with conductive contact regions associated with the cartridge 106 of FIG. 1 during insertion of the cartridge 106 of FIG. 1 into the instrument 102.

The main circuit board 204 is configured to support electrical communication, signal acquisition, power distribution, processing operations, and computational operations associated with the system 100 of FIG. 1. In certain embodiments, the main circuit board 204 comprises a printed circuit board including conductive routing structures, multilayer interconnect structures, grounding regions, shielding regions, power distribution pathways, connector interfaces, processor interfaces, analog front-end circuitry, wireless communication circuitry, memory interfaces, or combinations thereof. The main circuit board 204 may be fabricated using rigid printed circuit board materials, flexible printed circuit board materials, rigid-flex structures, ceramic circuit substrates, insulated metal substrates, or other electronic substrate structures suitable for supporting operation of the instrument 102.

In certain embodiments, the plurality of electronic chips and circuits 206 is associated with the operation of the power light 110. The plurality of electronic chips and circuits 206 may include illumination control circuitry, driver circuitry, voltage regulation circuitry, pulse width modulation circuitry, current limiting circuitry, indicator control logic, or other electronic systems configured to control visual operational indicators associated with the power light 110. The power light 110 may indicate power availability, charging status, communication activity, operational readiness, cartridge detection, assay execution status, warning conditions, fault conditions, or low-battery conditions associated with operation of the instrument 102.

The plurality of electronic chips and circuits 208 includes one or more microchips configured to control processing operations associated with the system 100 of FIG. 1. In certain embodiments, the plurality of electronic chips and circuits 208 includes microprocessors, microcontrollers, digital signal processors, field programmable gate arrays, system on chip devices, graphics processing units, tensor processing units, neural network accelerators, memory controllers, communication controllers, programmable logic devices, application specific integrated circuits, or combinations thereof. The plurality of electronic chips and circuits 208 may execute instructions configured to control cartridge interrogation, signal acquisition, analog to digital conversion, filtering, feature extraction, analyte classification, aggregation operations, confidence scoring operations, cloud communication operations, user interface operations, or result generation operations associated with the system 100.

In certain embodiments, the plurality of electronic chips and circuits 210 is associated with the operation of the auxiliary ports 112. The plurality of electronic chips and circuits 210 may include USB controller circuitry, Ethernet controller circuitry, serial communication circuitry, wireless communication circuitry, audio circuitry, charging circuitry, debugging circuitry, firmware update circuitry, or combinations thereof configured to support communication between the instrument 102 and one or more external systems. In certain embodiments, the auxiliary ports 112 communicate with mobile computing systems, laboratory systems, cloud computing systems, hospital systems, remote databases, or external computing platforms through wired or wireless communication channels.

The plurality of electronic chips and circuits 212 is associated with the operation of the power button 108. In certain embodiments, the plurality of electronic chips and circuits 212 includes switch monitoring circuitry, debounce circuitry, low power wake circuitry, interrupt generation circuitry, capacitive sensing circuitry, operational mode control circuitry, voltage monitoring circuitry, or reset circuitry configured to control activation, deactivation, initialization, standby operations, reset operations, calibration operations, or operational mode transitions associated with the instrument 102.

In certain embodiments, the plurality of electronic chips and circuits 214 is associated with the operation of the power connection port 116. The plurality of electronic chips and circuits 214 may include voltage regulators, buck converters, boost converters, charging controllers, battery management circuitry, protection circuitry, thermal monitoring circuitry, current limiting circuitry, AC to DC conversion circuitry, filtering circuitry, or power conditioning circuitry configured to regulate electrical power supplied to components contained within the instrument 102. In certain embodiments, the power connection port 116 is configured to interface with external power adapters, charging assemblies, docking stations, USB power systems, or other external power infrastructures associated with the operation of the system 100.

The lower housing 218 is configured to support and enclose internal electronic components associated with the operation of the system 100 of FIG. 1. In certain embodiments, the lower housing 218 includes structural mounting features, thermal dissipation structures, vibration isolation structures, airflow channels, fastening structures, connector alignment structures, or support features configured to mechanically support the main circuit board 204 and the plurality of electronic chips and circuits 206, 208, 210, 212, 214 and 216. The lower housing 218 may comprise polymeric materials, composite materials, metallic materials, thermally conductive materials, or combinations thereof configured to provide structural integrity and environmental protection associated with the operation of the instrument 102.

In certain embodiments, the instrument 102 communicates with the cloud network 120 of FIG. 1 configured to support remote processing, analytics, synchronization, storage operations, management operations, or machine learning operations associated with the system 100 of FIG. 1. The cloud network 120 of FIG. 1 may include cloud computing platforms, distributed computing systems, edge computing systems, virtualized computing systems, server systems, remote databases, or network management infrastructures configured to process operational data associated with the instrument 102 and the cartridge 106 of FIG. 1. The cloud network 120 of FIG. 1 may further support remote analyte classification operations, epidemiological monitoring operations, calibration management operations, remote reporting operations, user authentication operations, predictive maintenance operations, distributed machine learning operations, laboratory information system integration, or electronic medical record integration associated with the operation of the system 100.

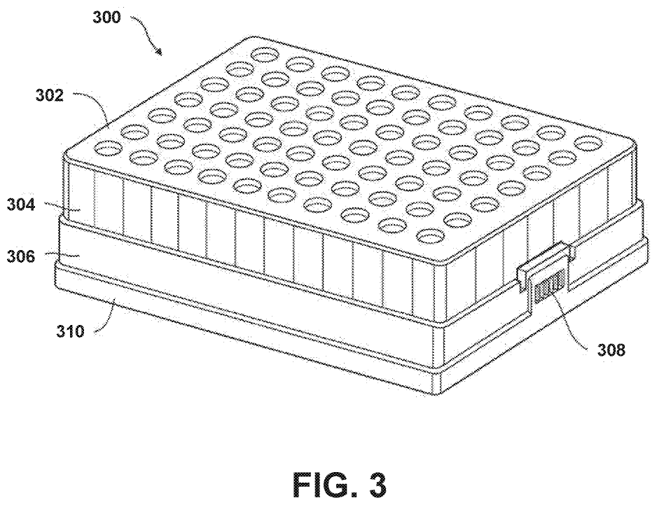

With reference to FIG. 3, the cartridge 106 of FIG. 1 is a multi-sensor cartridge 300 configured to receive a sample, distribute the sample across a plurality of sensing regions, and electrically interface with the instrument 102 of FIG. 1. In certain embodiments, the multi-sensor cartridge 300 includes an upper layer 302, a body portion 304, a sensor layer 306, a multi-sensor cartridge interface 308, and a lower cartridge housing 310. The various structures illustrated in FIG. 3 may cooperate to facilitate distributed sample delivery, redundant sensing operations, electrical signal generation, and analyte detection operations associated with the system 100 of FIG. 1.

The upper layer 302 is configured to receive one or more samples introduced into the multi-sensor cartridge 300. In certain embodiments, the upper layer 302 includes openings, ports, wells, reservoirs, membranes, channels, capillary structures, hydrophilic regions, hydrophobic barriers, or fluid directing structures configured to guide a sample into underlying regions of the multi-sensor cartridge 300. The upper layer 302 may further include sample loading regions, venting regions, metering regions, sealing structures, absorbent structures, overflow regions, or reagent regions associated with the operation of the multi-sensor cartridge 300.

Positioned beneath the upper layer 302 is the body portion 304, which is configured to distribute portions of the sample to sensing regions associated with the sensor layer 306. In certain embodiments, the body portion 304 includes microfluidic channels, capillary pathways, manifolds, branching flow structures, porous materials, wicking materials, diffusion structures, pressure-balancing structures, or fluidic routing networks configured to transport fluid from sample-receiving regions to sensing locations contained within the sensor layer 306.

The sensor layer 306 includes a plurality of sensing structures configured to generate electrical responses associated with analyte interactions. In certain embodiments, the sensor layer 306 comprises a flexible or rigid substrate containing individually addressable sensing elements arranged in rows, columns, arrays, matrices, concentric patterns, staggered configurations, or other geometric arrangements. The sensor layer 306 may include conductive traces, electrodes, sensing regions, contact pads, reference electrodes, counter electrodes, shielding structures, multiplexed routing structures, or interconnect structures configured to electrically couple sensing elements to the multi-sensor cartridge interface 308.

In certain embodiments, the sensor layer 306 is fabricated using printed circuit board fabrication processes, semiconductor fabrication processes, thin film deposition processes, photolithographic processes, screen printing processes, inkjet printing processes, flexible circuit fabrication processes, additive manufacturing processes, or combinations thereof. In certain preferred embodiments, the sensor layer 306 is fabricated using screen printing processes in which conductive inks are deposited onto a substrate through one or more patterned screens to form conductive traces, sensing electrodes, routing structures, contact pads, shielding regions, or conductive sensing structures associated with the sensing system.

The multi-sensor cartridge interface 308 is configured to electrically couple sensing elements contained within the sensor layer 306 to the electrical connectors 202 associated with the instrument 102 of FIG. 1. In certain embodiments, the multi-sensor cartridge interface 308 includes conductive pads, card edge contacts, plated interconnects, conductive elastomer interfaces, zero insertion force connector interfaces, flexible circuit structures, vias, through hole structures, multiplexed buses, or differential signaling pathways configured to communicate electrical signals between the multi-sensor cartridge 300 and the instrument 102 of FIG. 1.

The lower cartridge housing 310 is configured to provide structural support, dimensional stability, electrical interfacing, thermal management, or environmental protection associated with the operation of the multi-sensor cartridge 300. In certain embodiments, the lower cartridge housing 310 includes alignment features, retention features, cartridge insertion features, electrical contact regions, fiducial markers, barcode regions, QR code regions, RFID structures, or machine-readable identifiers associated with the cartridge 106.

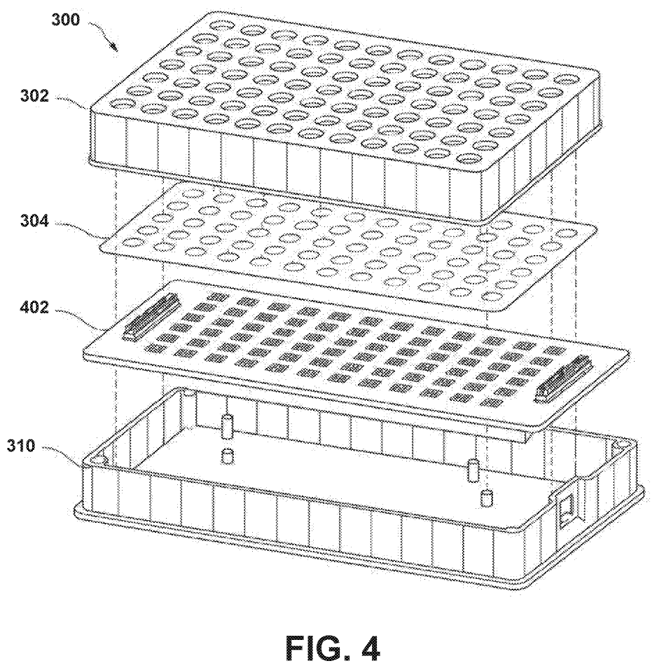

FIG. 4 shows an exploded view of one embodiment of the cartridge 106 of FIG. 1 as a multi-sensor cartridge 400 that includes an upper layer 302, a body portion fluidic gasket 304, a sensor array 402, a multi-sensor cartridge interface 308 of FIG. 3, and a lower cartridge housing 310 arranged in a multilayer cartridge architecture configured to receive a sample, distribute portions of the sample to multiple sensing regions, and electrically interface with the instrument 102 of FIG. 1 of the system 100 of FIG. 1. The structures illustrated in FIG. 4 may cooperate to facilitate distributed sample delivery, parallel sensor interrogation, redundant analyte detection operations, and electrical signal acquisition associated with operation of the system 100 of FIG. 1.

The upper layer 302 is configured to receive one or more samples introduced into the multi-sensor cartridge 300. In certain embodiments, the upper layer 302 includes openings, ports, wells, reservoirs, membranes, channels, capillary structures, hydrophilic regions, hydrophobic barriers, fluid-directing structures, or sample-loading regions configured to guide a sample into underlying regions of the multi-sensor cartridge 300. The upper layer 302 may further include venting structures, overflow regions, absorbent regions, reagent regions, sealing regions, metering structures, or alignment structures configured to facilitate controlled sample introduction and fluid distribution within the multi-sensor cartridge 300.

Positioned beneath the upper layer 302 is the body portion 304, which is configured to distribute portions of the sample to sensing regions associated with the sensor array 402. In certain embodiments, the body portion 304 includes fluidic gaskets, microfluidic channels, capillary pathways, manifolds, porous structures, wicking materials, branching flow structures, diffusion structures, pressure-balancing structures, reservoirs, or fluidic routing networks configured to transport fluid from sample-receiving regions to sensing locations contained within the sensor array 402. The body portion 304 may further include filtration regions, plasma separation regions, reagent mixing regions, amplification regions, incubation regions, denaturation regions, dilution regions, buffering regions, waste reservoirs, or reaction chambers configured to condition the sample, before interaction with sensing structures associated with the sensor array 402.

The sensor array 402 includes a plurality of sensing structures configured to generate electrical responses associated with analyte interactions. In certain embodiments, the sensor array 402 comprises a flexible or rigid substrate containing individually addressable sensing elements arranged in rows, columns, matrices, arrays, concentric arrangements, staggered configurations, or other geometric patterns. The sensor array 402 may include conductive traces, sensing electrodes, reference electrodes, counter electrodes, contact pads, shielding structures, routing structures, multiplexed addressing structures, or interconnect structures configured to electrically couple the sensing elements to the multi-sensor cartridge interface 308 of FIG. 3.

In certain embodiments, the sensor array 402 is fabricated using printed circuit board fabrication processes, semiconductor fabrication processes, thin film deposition processes, sputtering processes, vapor deposition processes, chemical vapor deposition, lithography, photolithographic processes, screen printing processes, inkjet printing processes, aerosol deposition processes, gravure printing, flexographic printing, offset printing, pad printing, spray coating, spin coating, slot-die coating, roll-to-roll print processing, laser patterning processes, flexible circuit fabrication processes, additive manufacturing processes, or combinations thereof. In certain preferred embodiments, the sensor array 402 is fabricated using screen printing processes in which conductive inks are deposited onto a substrate through one or more patterned screens to form conductive traces, sensing electrodes, routing structures, contact pads, shielding regions, or conductive sensing structures associated with the operation of the multi-sensor cartridge 300.

Each sensing structure within the sensor array 402 may include one or more active sensing regions configured to interact with one or more analytes. In certain embodiments, the active sensing regions include electrochemical sensing structures, impedance sensing structures, field effect transistor structures, capacitive sensing structures, resistive sensing structures, optical sensing structures, plasmonic sensing structures, piezoelectric sensing structures, magnetic sensing structures, or combinations thereof. The active sensing regions may further include immobilized probes, oligonucleotides, nucleic acid capture structures, antibodies, antigens, aptamers, enzymes, receptors, molecularly imprinted polymers, nanostructured coatings, conductive polymers, hydrogels, or biomolecular recognition materials configured to selectively interact with target analytes associated with the operation of the system 100 of FIG. 1.

In certain embodiments, subsets of sensing structures within the sensor array 402 are associated with a common analyte target to facilitate redundant sensing operations and aggregated signal analysis. Multiple sensing structures may therefore independently evaluate the same analyte and generate corresponding electrical responses associated with the analyte interaction. The plurality of electronic chips and circuits 208 of FIG. 2, the main circuit board 204 of FIG. 2, or processing systems associated with the cloud network 120 of FIG. 1 may aggregate responses across subsets of sensing structures to improve robustness, fault tolerance, reproducibility, confidence scoring, sensitivity, specificity, or resistance to localized defects, manufacturing variability, contamination, or sensor degradation.

The multi-sensor cartridge interface 308 of FIG. 3 is configured to electrically couple sensing structures contained within the sensor array 402 to the instrument 102 of FIG. 1. In certain embodiments, the multi-sensor cartridge interface 308 of FIG. 3 includes conductive pads, card edge contacts, plated interconnects, conductive elastomer interfaces, zero insertion force connector interfaces, flexible circuit structures, vias, through hole structures, multiplexed buses, differential signaling pathways, ribbon conductors, or shielded interconnect structures configured to communicate electrical signals between the multi-sensor cartridge 300 of FIG. 3 and the electrical connectors 202 of FIG. 2 associated with the instrument 102 of FIG. 1.

In certain embodiments, the multi-sensor cartridge interface 308 of FIG. 3 electrically interfaces with the main circuit board 204 of FIG. 2 and the plurality of electronic chips and circuits 208 of FIG. 2 associated with signal acquisition and processing operations performed by the instrument 102 of FIG. 1. The instrument 102 of FIG. 1 may apply excitation signals, bias voltages, current interrogation signals, impedance interrogation signals, pulse sequences, waveform interrogation signals, frequency sweeps, electrochemical interrogation sequences, or multiplexed interrogation patterns to sensing structures contained within the sensor array 402 and acquire corresponding electrical responses over time.

The lower cartridge housing 310 is configured to provide structural support, dimensional stability, thermal management, electrical interfacing, or environmental protection associated with the operation of the multi-sensor cartridge 300. In certain embodiments, the lower cartridge housing 310 includes alignment features, retention structures, cartridge insertion structures, electrical contact regions, fiducial markers, barcode regions, QR code regions, RFID structures, machine-readable identifiers, fastening regions, thermal management structures, or support structures configured to facilitate insertion and operation of the multi-sensor cartridge 300 of FIG. 3 within the instrument 102 of FIG. 1.

In certain embodiments, the lower cartridge housing 310 includes thermal management structures configured to influence operational temperatures associated with sensing operations performed by the sensor array 402. Such thermal management structures may include thermally conductive pathways, heat spreaders, embedded heaters, thermal sensors, insulation structures, phase change materials, thermal vias, thermoelectric components, or active thermal regulation systems configured to maintain operational temperatures associated with amplification reactions, denaturation processes, hybridization reactions, enzymatic reactions, or electrochemical sensing operations associated with the multi-sensor cartridge 300 of FIG. 3.

The structures associated with the multi-sensor cartridge 300 of FIG. 3 may operate together to facilitate distributed sample delivery, parallel sensor interrogation, redundant analyte detection, and aggregated signal acquisition operations associated with the system 100 of FIG. 1. A sample introduced into the upper layer 302 may travel through the body portion 304, become distributed across multiple sensing structures within the sensor array 402, and generate electrical responses communicated through the multi-sensor cartridge interface 308 of FIG. 3 to the electrical connectors 202 of FIG. 2, the main circuit board 204 of FIG. 2, and the plurality of electronic chips and circuits 208 of FIG. 2 associated with the instrument 102 of FIG. 1. The acquired electrical responses may subsequently be processed locally by the instrument 102 of FIG. 1 or communicated to the cloud network 120 for additional classification, aggregation, confidence scoring, machine learning analysis, reporting, epidemiological monitoring, or remote processing operations associated with the system 100 of FIG. 1.

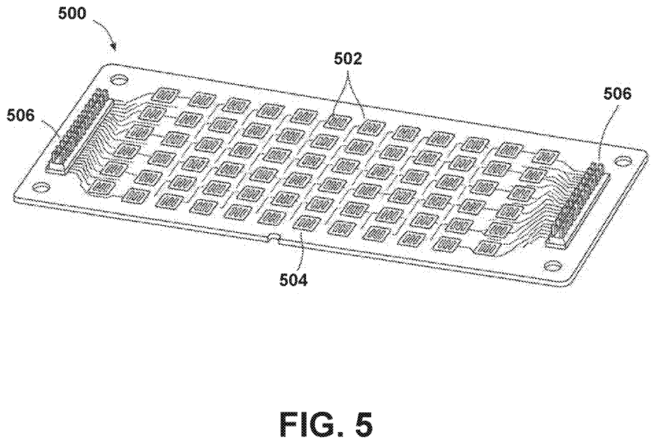

FIG. 5 shows the sensor array 402 of FIG. 4 of the cartridge 106 of FIG. 1 as an analyte detection sensor array 500. In certain embodiments, the analyte detection sensor array 500 includes a sequence of operations configured to acquire a sample, prepare the sample, introduce the sample into the cartridge 106 of FIG. 1, distribute portions of the sample throughout the multi-sensor cartridge 300 of FIG. 3, generate electrical responses using the sensor array 402 of FIG. 4, process acquired electrical signals, classify signal characteristics, aggregate sensor responses, and generate one or more analyte detection outputs associated with operation of the system 100 of FIG. 1. The analyte detection sensor array 500 may be implemented using the instrument 102 of FIG. 1, the cartridge 106 of FIG. 1, the cloud network 120 of FIG. 1, the main circuit board 204 of FIG. 2, the plurality of electronic chips and circuits 208 of FIG. 2, or combinations thereof.

In certain embodiments, the analyte detection sensor array 500 begins by processing a sample that is collected from a subject, environmental source, industrial process, laboratory specimen, agricultural source, or testing environment. The collected sample may comprise blood, plasma, serum, saliva, urine, tissue extracts, nasal swabs, throat swabs, wound swabs, sputum, cerebrospinal fluid, environmental water samples, food extracts, agriculture extracts, chemical solutions, airborne particulate samples, industrial fluids, or combinations thereof. Sample acquisition may be performed manually, semi-automatically, or automatically using collection tubes, capillary devices, syringes, pipettes, swabs, microfluidic sampling systems, robotic liquid handling systems, vacuum systems, absorbent materials, or integrated sampling interfaces associated with the cartridge 106 of FIG. 1.

Following sample acquisition, the sample may include one or more sample preparation operations configured to condition the sample before introduction into the cartridge 106 of FIG. 1. In certain embodiments, sample preparation operations include plasma separation, filtration, centrifugation, dilution, concentration, lysis, buffering, reagent mixing, amplification preparation, denaturation, purification, extraction, washing, incubation, heating, cooling, or stabilization operations. The sample preparation operations may be performed externally using laboratory equipment or internally using structures associated with the upper layer 302 of FIG. 3, the body portion 304 of FIG. 3, or the lower cartridge housing 310 of FIG. 3 of the multi-sensor cartridge 300 of FIG. 3.

In certain embodiments, the sample for the analyte detection sensor array 500 can be introduced into the multi-sensor cartridge 300 of FIG. 3 through one or more sample ports, wells, reservoirs, capillary loading regions, puncturable membranes, injection interfaces, pipette interfaces, or microfluidic loading channels associated with the upper layer 302 of FIG. 3. The sample loading operation may utilize passive capillary action, pressure-driven flow, vacuum-assisted flow, centrifugal flow, electroosmotic flow, piezoelectric pumping systems, pneumatic actuation systems, electrowetting systems, or gravity assisted transport systems configured to move the sample into sensing regions associated with the sensor array 402 of FIG. 4.

In certain embodiments, the analyte detection sensor array 500 can be initiated by a sample distribution operation in which the sample is distributed across a plurality of sensor elements 502 contained within the sensor array 402 of FIG. 4. The sample distribution operation may utilize microfluidic channels, branching manifolds, porous membranes, hydrophilic pathways, diffusion structures, flow balancing channels, absorbent structures, or pressure equalization systems associated with the body portion 304 of FIG. 3 is configured to distribute portions of the sample to multiple sensor elements 502 substantially simultaneously or according to predefined sequencing operations. Alternative embodiments may include fluidic valves, microvalves, burst valves, capillary stop structures, phase change valves, dissolvable barriers, or programmable flow control systems configured to regulate sample movement throughout the multi-sensor cartridge 300 of FIG. 3.

In certain embodiments, the analyte detection sensor array 500 includes an analyte interaction stage during which one or more analytes within the sample interact with active sensing regions associated with the sensor elements 502 distributed throughout the sensor array 402 of FIG. 4. The sensor elements 502 may include electronic sensing structures, electrochemical sensing structures, impedance sensing structures, field effect transistor structures, capacitive sensing structures, resistive sensing structures, optical sensing structures, plasmonic sensing structures, piezoelectric sensing structures, magnetic sensing structures, or combinations thereof. The sensor elements 502 may further include immobilized antibodies, antigens, nucleic acid probes, oligonucleotides, aptamers, enzymes, receptors, nanostructured coatings, conductive polymers, molecularly imprinted polymers, hydrogels, biomolecular recognition materials, or chemically functionalized surfaces configured to selectively interact with target analytes associated with operation of the system 100 of FIG. 1.

During the analyte interaction stage, the analyte detection sensor array 500 may have one or more environmental control operations configured to maintain operational conditions associated with sensing operations performed by the sensor array 402 of FIG. 4. Such environmental control operations may include temperature regulation, humidity regulation, vibration isolation, electromagnetic shielding, pressure regulation, atmospheric control, thermal cycling, reagent hydration control, ion control, pH measurement or airflow management operations. Temperature regulation may be performed using resistive heating elements, thermoelectric devices, infrared heating systems, thermal spreaders, phase change materials, cooling systems, thermal sensors, or feedback-controlled thermal management circuitry associated with the instrument 102 of FIG. 1, the main circuit board 204, or the lower cartridge housing 310 of FIG. 3.

Following analyte interaction, the analyte detection sensor array 500 includes a signal acquisition operation in which electrical responses generated by the sensor elements 502 are measured over time. In certain embodiments, signal acquisition includes acquisition of electrical current measurements, voltage measurements, impedance measurements, resistance measurements, capacitance measurements, frequency domain measurements, transient waveform measurements, pulse responses, electrochemical response curves, or combinations thereof. Signal acquisition may be performed using analog front-end circuitry associated with the plurality of electronic chips and circuits 208 of FIG. 2 and the main circuit board 204 of FIG. 2.

The signal acquisition operation may further include application of excitation conditions to the sensor elements 502 through conductive routing structures 504 and electrical interface connectors 506 associated with the sensor array 402 of FIG. 4. Such excitation conditions may include direct current bias signals, alternating current excitation signals, sinusoidal waveforms, square wave pulses, impedance spectroscopy waveforms, frequency sweeps, voltage pulses, current injection sequences, differential signaling patterns, or programmable interrogation waveforms. In certain embodiments, different sensor elements 502 receive differing excitation conditions depending on analyte type, sensor chemistry, operating mode, calibration state, or environmental conditions associated with operation of the system 100 of FIG. 1.

In certain embodiments, the analyte detection sensor array 500 includes one or more signal preprocessing 704 operations configured to condition acquired sensor data before classification and aggregation operations are performed. The preprocessing 704 operations may include baseline normalization operations, drift compensation operations, filtering operations, smoothing operations, transient rejection operations, interpolation operations, decimation operations, feature extraction operations, signal averaging operations, derivative calculations, frequency domain analysis operations, outlier rejection operations, or adaptive thresholding operations. The preprocessing 704 operations may compensate for environmental variability, electrical interference, manufacturing tolerances, sensor drift, thermal effects, humidity effects, or assay specific variability associated with operation of the sensor array 402 of FIG. 4.

Following preprocessing 704 operations, the analyte detection sensor array 500 includes one or more signal classification operations configured to classify acquired electrical responses generated by the sensor elements 502. In certain embodiments, classification operations are performed using rules-based systems, machine learning models, neural networks, support vector machines, Bayesian classifiers, random forest models, ensemble classifiers, statistical inference systems, fuzzy logic systems, or combinations thereof executed by the plurality of electronic chips and circuits 208 or processing systems associated with the cloud network 120 of FIG. 1. The classification operations may classify signals as strong positive signals, weak positive signals, strong negative signals, weak negative signals, invalid signals, noisy signals, saturated signals, inconclusive signals, or control failure signals based on electrical response characteristics associated with the sensor elements 502.

In certain embodiments, the analyte detection sensor array 500 includes one or more aggregation operations configured to aggregate signal classifications or signal characteristics across subsets of sensor elements 502 associated with common analyte targets. The aggregation operations may include weighted voting systems, confidence scoring systems, statistical averaging operations, probabilistic fusion operations, consensus analysis operations, temporal correlation operations, machine learning aggregation operations, or Bayesian inference systems. The aggregation operations may generate analyte determinations based on combined sensor behavior rather than reliance upon individual sensor outputs generated by isolated sensor elements 502.

The analyte detection sensor array 500 may further include one or more confidence-scoring operations configured to generate confidence values associated with analyte determinations. Confidence scores may be based on the number of responsive sensor elements 502, signal magnitude consistency, waveform similarity, temporal agreement, signal persistence, environmental compensation factors, calibration history, sensor weighting parameters, machine learning confidence outputs, or combinations thereof. The confidence scores may be represented numerically, probabilistically, graphically, textually, colorimetrically, or through multilevel categorical indicators presented on the display screen 104 of FIG. 1 or transmitted to the cloud network 120 of FIG. 1.

In certain embodiments, the analyte detection sensor array 500 includes one or more quality assurance operations configured to evaluate assay integrity, cartridge integrity, calibration validity, sensor performance, environmental stability, reagent validity, or data integrity associated with the multi-sensor cartridge 300 of FIG. 3 and the sensor layer 306 of FIG. 3. Such quality assurance operations may include evaluation of reference sensing regions, calibration sensing regions, internal controls, reagent controls, environmental sensors, signal integrity metrics, temperature measurements, ionic measurements, pH measurements, humidity measurements, timing validation operations, or cloud-based validation systems associated with the cloud network 120 of FIG. 1. In certain embodiments, the analyte detection sensor array 500 generates invalid or inconclusive outputs in response to detected abnormalities or control failures associated with the sensor elements 502.

The analyte detection sensor array 500 may further include one or more cloud communication operations configured to transmit acquired sensor data, extracted features, classification outputs, confidence scores, cartridge identifiers, assay metadata, calibration information, or diagnostic information to remote computing systems associated with the cloud network 120 of FIG. 1. The cloud network 120 of FIG. 1 may include cloud servers, distributed analytics systems, machine learning training systems, hospital systems, laboratory information systems, epidemiological monitoring systems, electronic medical record systems, mobile applications, or remote databases. In certain embodiments, the cloud network 120 of FIG. 1 performs additional classification operations, analytics operations, machine-learning retraining operations, mutation-monitoring operations, epidemiological analysis operations, or software update operations associated with the operation of the system 100 of FIG. 1.

Following completion of classification and aggregation operations, the analyte detection sensor array 500 includes a result generation operation configured to generate one or more outputs associated with analyte detection operations performed by the system 100 of FIG. 1. The generated outputs may include positive or negative determinations, mutation detection results, quantitative analyte concentrations, confidence scores, waveform visualizations, temporal graphs, epidemiological alerts, assay status indicators, quality control indicators, calibration notifications, or historical comparison data. The outputs may be displayed locally on the display screen 104 of FIG. 1 of the instrument 102 of FIG. 1, transmitted through the auxiliary ports 112 of FIG. 1, uploaded to the cloud network 120 of FIG. 1, printed on reports, integrated into electronic medical record systems, or transmitted to external systems through secure communication channels.

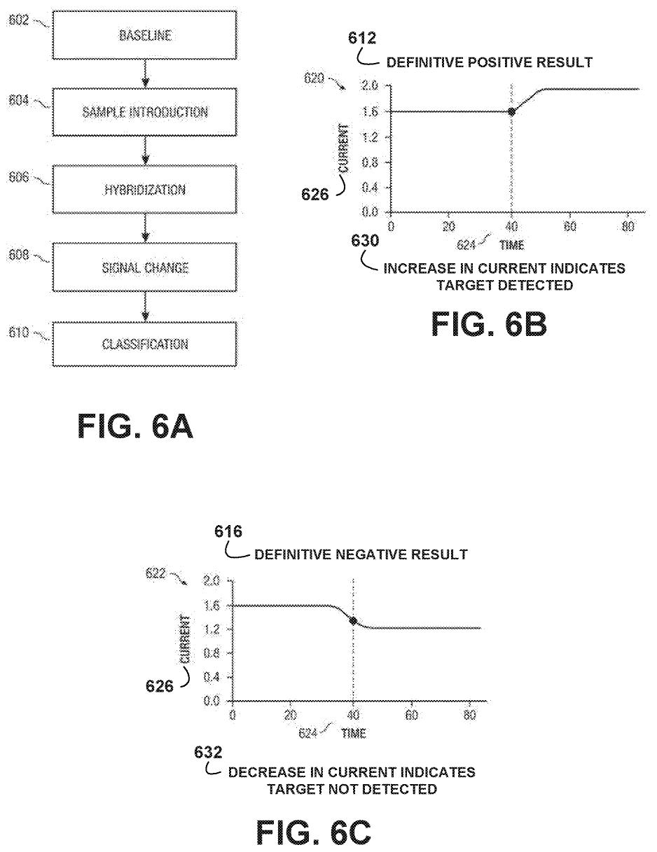

FIG. 6A illustrates a representative electrical signal response. FIG. 6A depicts example electrical response profiles generated by one or more sensing regions associated with a sensor array during exposure to a sample containing one or more analytes or lacking one or more analytes.

FIG. 6A is a baseline 602, which shows changes in the baseline 602 in an electrical response when a sample introduction 604 is made. The sample has been processed with hybridization 606. Upon the start of an electrical charge, the resulting signal shows a signal change 608 from the baseline 602. The signal change 608 is processed with a classification 610 process to identify the nature of the change and possible detection of an analyte, dependent on the classification 610.

In certain embodiments, the illustrated signal responses represent time-domain electrical measurements acquired from electronic sensing structures, electrochemical sensing structures, impedance sensing structures, current 626 of FIG. 6B sensing structures, capacitive sensing structures, transistor-based sensing structures, or other sensor architectures configured to generate measurable electrical outputs associated with analyte interactions. The representative signal responses may be utilized by one or more classification systems, aggregation systems, machine learning systems, or confidence-scoring systems to determine the presence, absence, concentration, mutation state, or other characteristics associated with one or more analytes.

FIG. 6B illustrates representative examples of positive electrical response characteristics evaluated over time. FIG. 6C illustrates representative examples of negative electrical response characteristics evaluated over time. In alternative embodiments, the signal responses illustrated in FIG. 6B includes a definitive positive signal 612 response and the signal responses illustrated in FIG. 6C includes a definitive negative signal 616 response. Referring to both FIG. 6B and FIG. 6C, the definitive positive signal profile 620 response may correspond to electrical behavior associated with analyte binding, target interaction, electrochemical reaction activity, impedance variation, charge transfer activity, molecular hybridization, catalytic activity, or other measurable sensing events occurring at one or more sensing regions. The definitive negative signal profile 622 response may correspond to electrical behavior associated with the absence of analyte interaction, suppressed electrochemical activity, inverse electrical behavior, baseline stabilization, lack of target binding, or absence of measurable sensing activity associated with a target analyte.

Referring to both FIG. 6B and FIG. 6C, the representative electrical signal responses may be plotted as electrical current 626 versus time 624, voltage versus time 624, impedance versus time 624, resistance versus time 624, capacitance versus time 624, phase response versus time 624, frequency-domain response versus time 624, or other measurable electrical characteristics associated with the operation of the sensing system. In defined embodiments, the horizontal axis represents elapsed time 624 during an assay operation, while the vertical axis represents an electrical parameter acquired from one or more sensing regions. Alternative embodiments may utilize logarithmic scales, normalized scales, transformed feature spaces, multidimensional signal spaces, or probabilistic response spaces associated with classification operations. A signal profile 620 shows a current 626 flow increase by 0.4 units of current over time 624, where an increase in current indicates a target detected 630.

The definitive negative signal 616 response may include one or more decreasing electrical response characteristics over time 624. In certain embodiments, the negative signal response includes an initial baseline region followed by a transition region exhibiting decreasing electrical current 626 magnitude, decreasing slope characteristics, transient electrical changes, nonlinear response behavior, exponential diminishing behavior, or unstabilized elevated response levels. The transition region may correspond to the lack of onset of analyte binding, decreasing of electronic reactions, electrochemical reactions, non-hybridization events, non-conductive pathway formation, a lack of charge-transfer reactions, molecular lack of recognition events, catalytic lack of amplification events, or other physical or chemical non-interactions occurring at the sensing regions.

In certain embodiments, the positive signal response includes a stepwise transition region in which the electrical signal rapidly changes from a baseline level to an elevated level. The stepwise transition may correspond to threshold activation behavior, cooperative binding behavior, amplification reactions, electronic switching events, electrochemical switching events, phase transitions, or other abrupt electrical response phenomena associated with analyte interactions. Alternative embodiments may include gradual increases, sigmoidal transitions, oscillatory increases, multiphasic response behavior, delayed activation responses, transient spikes, pulse responses, or combinations thereof.

The definitive positive signal 612 response may further include a stabilization region in which the electrical signal approaches a substantially stable elevated value. In certain embodiments, the stabilization region corresponds to equilibrium binding conditions, stabilized electrochemical conditions, completed amplification reactions, stabilized charge transfer activity, or saturation of active sensing regions. The classification systems may evaluate the magnitude, duration, stability, slope characteristics, temporal persistence, derivative behavior, or waveform consistency associated with the stabilization region during classification operations.

The definitive negative signal 616 response may include one or more decreasing electrical response characteristics over time 624. In certain embodiments, the negative signal response includes an initial baseline region followed by a transition region exhibiting decreasing electrical current 626 magnitude, decreasing slope characteristics, suppression behavior, inverse waveform characteristics, or stabilized lower-level responses. The negative response behavior may correspond to the absence of analyte binding, inhibitory interactions, electronic suppression, electrochemical suppression, lack of conductive pathway formation, degradation behavior, reference-state conditions, or non-reactive sensing conditions.

In certain embodiments, the definitive negative signal response 616 includes a downward transition region followed by a stabilized lower-level electrical response. The downward transition may correspond to decay of transient excitation effects, relaxation behavior, baseline stabilization, inhibitory electrochemical activity, environmental compensation behavior, or absence of target-induced electrical changes. Alternative embodiments may include substantially flat responses, low-noise baseline responses, oscillatory negative responses, delayed decay responses, inverse transient behavior, or multiphasic negative waveform patterns.

The representative signal responses illustrated in FIG. 6B may further include one or more weak positive signal profiles, weak negative signal profiles, noisy signal profiles, unstable signal profiles, invalid signal profiles, or inconclusive signal profiles, not necessarily illustrated in simplified form. Weak positive signals may exhibit lower amplitude increases, delayed activation characteristics, partial response behavior, intermittent response patterns, mixed increasing and decreasing segments, oscillatory behavior, or incomplete stabilization behavior. Weak negative signals may exhibit partial suppression behavior, low-amplitude decay characteristics, unstable baselines, or mixed polarity transitions. In alternative embodiments, classification operations are performed based on changes in one or more electrical characteristics over time 624, including current 626 magnitude, impedance magnitude, voltage magnitude, capacitance characteristics, waveform shape characteristics, derivative characteristics, temporal characteristics, spectral characteristics, or combinations thereof.

In certain embodiments, invalid signal responses correspond to substantially flat signals, saturated signals, disconnected sensor responses, corrupted waveforms, unstable baselines, excessive electrical noise, drift-dominated responses, environmental interference, incomplete acquisitions, sensor failures, or calibration failures. The classification systems may identify invalid signal responses by evaluating signal variance, temporal instability, derivative discontinuities, saturation levels, signal-to-noise ratios, baseline integrity, waveform consistency, or other measurable electrical characteristics.

The representative signal responses of FIG. 6B may be acquired from one or more sensing regions distributed throughout a sensor array. In certain embodiments, multiple sensing regions assigned to a common analyte generate corresponding electrical response profiles that are subsequently aggregated by one or more processing systems. The processing systems may evaluate agreement between signal profiles, waveform similarity, temporal correlation, signal magnitude consistency, stabilization characteristics, derivative behavior, frequency-domain similarity, or classification consistency across the subset of sensing regions associated with the analyte.

In certain embodiments, the representative signal responses are analyzed using one or more preprocessing 704 systems prior to classification operations. The preprocessing 704 systems may perform baseline normalization, offset correction, drift compensation, filtering operations, smoothing operations, derivative calculations, interpolation operations, frequency-domain transformations, spectral analysis operations, or adaptive thresholding operations.

Preprocessing 704 produces baseline current flows through semiconducting circuits. The sample introduces nucleic acids that binds to surface probes, hybridization changes sensor resistance, current indicates target presence or absence, and an increase in current =positive result; a decrease in current =negative result. The following tables show the ranges in definitive positive result 612 and definitive negative result 616. Examples show a definitive positive result 612 on the table with a current increase after a specified period of time, and show a definitive negative result 616 signal profile 620 on the table with a current signal profile 622 decrease after a specified period of time. An increase in current indicates target detected 630, and a decrease in current indicates target not detected 632.

Such preprocessing operations may reduce electrical noise, compensate for environmental variability, compensate for sensor aging, compensate for manufacturing variation, tolerances, or improve classification robustness.

The classification systems associated with FIG. 6 may include rules-based classifiers, statistical classifiers, neural networks, support vector machines, Bayesian classifiers, random forest models, ensemble classifiers, convolutional neural networks, recurrent neural networks, transformer architectures, fuzzy logic systems, hidden Markov models, or combinations thereof. The classification systems may evaluate signal shape characteristics, temporal progression characteristics, slope values, derivative behavior, peak magnitudes, settling characteristics, oscillatory features, spectral characteristics, or multidimensional feature vectors associated with the representative signal responses.

In certain embodiments, the representative signal responses are converted into feature vectors for machine learning analysis. The feature vectors may include normalized current 626 values, derivative sequences, Fourier coefficients, wavelet coefficients, temporal correlation values, entropy measurements, variance measurements, spectral densities, impedance characteristics, transient durations, or other measurable parameters associated with the signal responses. The feature vectors may subsequently be evaluated using trained machine learning models configured to classify analyte states or predict assay outcomes.

The signal responses illustrated in FIG. 6B may further be utilized during calibration operations associated with the sensing system. In certain embodiments, known positive control samples and known negative control samples are utilized to generate calibration response profiles corresponding to definitive positive and definitive negative outcomes. The calibration response profiles may be stored locally within an instrument, remotely within cloud computing systems, or within distributed machine learning databases. Subsequent assay responses may be compared against the calibration response profiles to improve classification accuracy, confidence-scoring accuracy, or environmental compensation performance.

In certain embodiments, the representative signal responses may vary depending on analyte concentration, sample composition, environmental conditions, sensor chemistry, interrogation waveform characteristics, amplification conditions, reagent conditions, or cartridge-specific parameters. Certain embodiments may therefore utilize adaptive threshold systems, dynamic classification boundaries, machine learning retraining systems for signal models, environmental compensation systems, or analyte-specific calibration models configured to accommodate variability in the representative signal responses.

FIG. 6B illustrates a representative example of positive and negative electrical response characteristics evaluated over time.

The representative signal responses may further be evaluated using temporal analysis systems configured to identify persistence characteristics, stabilization times, delayed activation events, transient oscillations, recovery behavior, hysteresis behavior, relaxation characteristics, or kinetic properties associated with analyte interactions. Such temporal analysis systems may improve differentiation between true analyte-associated responses and noise-induced or environmentally induced signal artifacts.

In alternative embodiments, the representative signal responses are aggregated across multiple sensing regions assigned to a common analyte target. The aggregation systems may perform weighted voting operations, consensus analysis operations, confidence scoring operations, Bayesian fusion operations, statistical averaging operations, neural network aggregation operations, or probabilistic inference operations. A system-level analyte determination may therefore be generated based on combined electrical response characteristics distributed across multiple sensors rather than reliance upon a single sensor response.

The representative signal responses illustrated in FIG. 6B may additionally be utilized for cloud-based analytics, epidemiological monitoring, mutation tracking, longitudinal monitoring, assay optimization, quality assurance operations, determining clinical treatment or machine learning retraining operations. Cloud computing systems may aggregate representative signal responses acquired from geographically distributed instruments to identify population-level patterns, environmental influences, mutation-specific waveform signatures, cartridge manufacturing trends, or calibration drift characteristics.

Alternative embodiments associated with FIG. 6B may utilize electrochemical current 626 response profiles, electronic current, impedance spectroscopy response profiles, transistor threshold voltage shifts, capacitive transient responses, optical intensity curves, fluorescence decay curves, magnetic response signatures, acoustic resonance responses, or combinations thereof. Certain embodiments may utilize commercially available analog front-end integrated circuits, digital signal processors, electrochemical analyzers, impedance analyzers, machine learning software frameworks, cloud analytics platforms, or waveform processing systems adapted to perform the acquisition, analysis, and classification operations associated with the representative signal responses described herein.

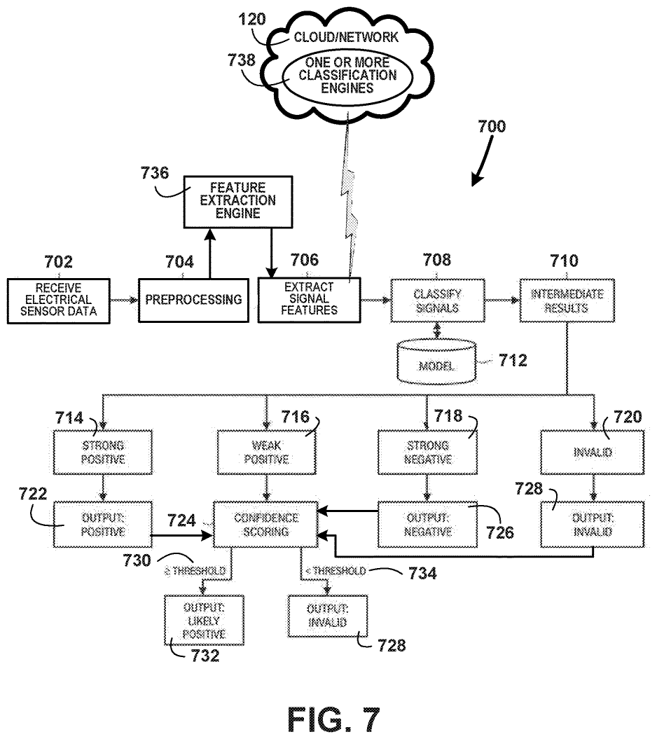

In certain embodiments, the representative signal responses illustrated in FIG. 6B form part of an integrated classification architecture configured to transform electrical response behavior generated across a plurality of sensing regions into analyte determinations based on waveform analysis, temporal analysis, signal aggregation, confidence scoring, and machine-learning inference operations. The interaction between the sensing regions, signal acquisition systems, preprocessing 704 systems, classification engines 738 of FIG. 7, aggregation systems, and confidence scoring systems enables robust analyte detection using distributed electrical response profiles associated with definitive positive and definitive negative assay outcomes.

FIG. 7 illustrates a system 100 of FIG. 1 for processing sensor data and generating classification outputs. The processing system is configured to receive electrical sensor data 702, acquire the data, extract signal features 706, classify signals 708 of sensor responses, aggregate classification results in a model 712 database across multiple sensing regions, and intermediate results 710, including strong positive 714, weak positive 716, strong negative 718, and invalid 720. The intermediate results 710 are initially classified as outputs.

The strong positive 714 signal produced an output positive 722 confidence score, the strong negative 718 signal produced an output negative 726 confidence score, and the invalid 720 signal produced an output invalid 728 confidence score. The weak positive 716 is also analyzed for the confidence scoring 724 and shows an example of the scoring with a comparison of the signal profile readings when≥threshold 730. In this instance, an output likely positive 732 confidence scoring 724, while a signal profile reading<threshold 734 is scored as an output invalid 728 rating.

The process is used to generate confidence scores, and output one or more analyte determinations or related analytical results. In alternative embodiments, the processing system 700 is implemented using one or more processors associated with a reader instrument, cloud computing platform, distributed computing system, edge computing architecture, mobile computing device, server infrastructure, or combinations thereof. The processing system may be configured to process electrical responses associated with electronic sensing systems, electrochemical sensing systems, impedance sensing systems, transistor-based sensing systems, capacitive sensing systems, optical sensing systems, magnetic sensing systems, acoustic sensing systems, or combinations thereof.

In certain embodiments, the processing system receives sensor data from a plurality of sensors distributed throughout a sensor sheet or cartridge assembly. The sensor data may include electrical current measurements, voltage measurements, impedance measurements, capacitance measurements, resistance measurements, frequency-domain measurements, electrochemical response curves, transient waveforms, phase measurements, pulse response data, or combinations thereof acquired over time. The sensor data may be generated in response to analyte interactions occurring at sensing regions configured with analyte-specific probes, receptors, antibodies, nucleic acid capture structures, oligonucleotides, aptamers, conductive polymers, nanostructured sensing surfaces, or other active sensing materials.

The processing system includes a data acquisition interface configured to receive sensor data from one or more analog front-end systems associated with a reader instrument or external sensing platform. In certain embodiments, the data acquisition interface includes analog-to-digital converters, serial communication interfaces, wireless communication modules, multiplexing interfaces, memory buffers, direct memory access controllers, digital signal processors, or network communication interfaces configured to receive sensor data from local or remote sources. The acquired sensor data may be streamed continuously, transferred in batches, transferred asynchronously, transferred synchronously, or transferred according to predefined interrogation sequences.

In certain embodiments, the processing system includes a preprocessing engine configured to condition the acquired sensor data before feature extraction and classification operations are performed. The preprocessing engine may include filtering systems, baseline normalization systems, offset correction systems, drift compensation systems, noise reduction systems, smoothing systems, interpolation systems, resampling systems, transient rejection systems, adaptive threshold systems, derivative calculation systems, spectral analysis systems, or data normalization systems.

The preprocessing engine may implement finite impulse response filters, infinite impulse response filters, moving average filters, Kalman filters, Savitzky-Golay filters, Gaussian filters, Wiener filters, low-pass filters, high-pass filters, notch filters, adaptive filtering systems, or machine learning-based denoising systems configured to reduce electrical noise or environmental interference associated with the acquired sensor data. In alternative embodiments, the preprocessing engine compensates for thermal drift, humidity variation, electromagnetic interference, sensor aging, reagent degradation, manufacturing variability, cartridge variability, or power supply fluctuations.

In certain embodiments, the preprocessing engine performs baseline correction operations configured to normalize sensor responses relative to initial baseline conditions. The baseline correction operations may remove offsets, normalize signal amplitudes, compensate for sensor-to-sensor variability, or align sensor responses before feature extraction operations. Certain embodiments may further perform temporal alignment operations configured to synchronize sensor responses acquired from different sensing regions or different cartridges.

Following preprocessing 704, the processing system includes a feature extraction engine 736 configured to derive measurable signal characteristics associated with the acquired sensor data. The feature extraction engine 736 may calculate slope values, derivative values, transient durations, peak amplitudes, integrated signal areas, impedance values, frequency-domain features, waveform shape parameters, entropy values, variance measurements, temporal correlation coefficients, rise times, settling times, oscillatory characteristics, spectral features, relaxation characteristics, phase response characteristics, or multidimensional feature vectors associated with the sensor responses.

In certain embodiments, the feature extraction engine 736 performs signal transformation operations, including Fourier transforms, wavelet transforms, Hilbert transforms, Laplace transforms, principal component analysis operations, singular value decomposition operations, manifold learning operations, autoencoder transformations, clustering transformations, dimensionality reduction operations, or nonlinear mapping operations configured to generate transformed feature representations associated with the acquired sensor data.

The feature extraction engine 736 may further generate feature vectors corresponding to individual sensing regions, groups of sensing regions, analyte-specific sensor subsets, reference sensing regions, or aggregated sensor populations. The generated feature vectors may subsequently be stored within local memory systems, transmitted to cloud/network 120 of FIG. 1 computing systems, utilized for machine learning inference operations, or provided to one or more classification engines 738.

In certain embodiments, the processing system includes one or more classification engines 738 configured to classify signals 708 responses based on the extracted features and acquired signal characteristics. The classification engines 738 may include rules-based classifiers, statistical inference systems, neural networks, convolutional neural networks, recurrent neural networks, transformer models, support vector machines, Bayesian classifiers, hidden Markov models, random forest classifiers, gradient boosting systems, ensemble learning systems, fuzzy logic systems, logistic regression systems, nearest neighbor systems, or combinations thereof.

The classification engines 738 may classify signals 708 responses into one or more categories, including strong positive 714 classifications, weak positive 716 classifications, strong negative 718 classifications, invalid 720 classifications, inconclusive classifications, noisy classifications, unstable classifications, saturated classifications, drift-dominated classifications, or control-failure classifications, indicating an output: invalid 720. In certain embodiments, the classification categories correspond to electrical behaviors associated with analyte presence, analyte absence, incomplete reactions, contamination, environmental interference, sensor degradation, reagent instability, or calibration abnormalities.

A strong positive classification 714 may correspond to increasing current behavior, elevated impedance behavior, stabilized positive waveform characteristics, transient activation sequences, analyte-associated electrochemical responses, or machine learning confidence values exceeding one or more thresholds. A weak positive 716 classification may correspond to lower-magnitude responses, delayed activation responses, partial waveform signatures, intermittent positive behaviors, oscillatory responses, or mixed-polarity characteristics. A strong negative 718 classification may correspond to decreasing current behavior, suppressed electrical activity, inverse waveform characteristics, stabilized low-level responses, or absence of analyte-associated response patterns. An invalid 720 classification may correspond to saturated responses, substantially flat responses, corrupted waveforms, excessive noise conditions, unstable baselines, disconnected sensors, or incomplete acquisition events.

In certain embodiments, the classification engines 738 access one or more model 712 databases configured to store trained machine learning models, classification parameters, calibration data, threshold values, waveform templates, analyte-specific feature sets, sensor weighting parameters, environmental compensation models, or historical sensor datasets. The model 712 databases may be stored locally within a reader instrument, remotely within cloud computing systems, within distributed database systems, or within secure edge-computing infrastructures. The classification engines 738 may retrieve updated machine learning models, calibration profiles, mutation signatures, epidemiological datasets, or assay-specific parameters from remote cloud systems during operation.

The model 712 databases may further store analyte-specific waveform signatures, mutation-specific response patterns, calibration curves, sensor aging profiles, environmental compensation data, historical assay outcomes, or population-level datasets aggregated from geographically distributed sensing systems. In certain embodiments, the processing system utilizes continuous learning operations configured to update stored models based on newly acquired sensor data and validated assay outcomes.

Following classification operations, the processing system 700 includes one or more aggregation engines configured to aggregate classifications or signal characteristics across multiple sensing regions associated with a common analyte target. In certain embodiments, the aggregation engines perform weighted voting operations, statistical averaging operations, consensus analysis operations, Bayesian fusion operations, neural network aggregation operations, temporal correlation analysis operations, confidence fusion operations, or probabilistic inference operations.

The aggregation engines may evaluate agreement between sensing regions based on waveform similarity, temporal consistency, signal magnitude correlation, derivative similarity, classification agreement, stabilization characteristics, frequency-domain similarity, or machine learning confidence values. Certain embodiments may assign differing weighting factors to different sensing regions based on calibration history, sensor reliability, manufacturing tolerances, environmental conditions, signal quality metrics, sensor age, or analyte-specific performance characteristics.

In certain embodiments, the processing system includes a confidence-scoring 724 engine configured to generate confidence values associated with analyte determinations. The confidence scoring 724 engine may calculate confidence values based on the number of sensors classified as positive, signal magnitude consistency, temporal agreement between sensing regions, waveform similarity, signal persistence, feature vector similarity, calibration accuracy, environmental compensation factors, machine learning confidence outputs, or combinations thereof. In alternative embodiments, one or more strong negative classifications reduce, suppress, outweigh, or override weak positive classifications during analyte determination. The outputs include, output: positive 722, output: negative 726, confidence score≥threshold 730, output: likely positive 732, confidence score<threshold 734, and confidence scoring 724, output: invalid 728.

The confidence scoring 724 engine may generate numerical confidence scores, probabilistic confidence values, categorical confidence levels, graphical confidence indicators, color-coded confidence outputs, or multi-dimensional confidence representations. In alternative embodiments, analyte presence is determined when one or more strong positive classifications are detected within a subset of sensors associated with the analyte. Alternative embodiments may determine analyte presence when multiple weak positive classifications collectively exceed one or more confidence thresholds despite the absence of any individual strong positive classification.

In certain embodiments, the aggregation engines and confidence scoring 724 engine apply override logic configured to resolve conflicting sensor outputs. For example, strong negative classifications may reduce or override weak positive classifications under certain operational conditions. Alternative embodiments may utilize probabilistic balancing systems, Bayesian reconciliation systems, neural network inference systems, fuzzy logic systems, or weighted consensus systems configured to resolve conflicting classifications across multiple sensing regions.

The processing system may further include one or more temporal analysis engines configured to evaluate sensor behavior over time. The temporal analysis engines may identify transient activation sequences, stabilization behavior, delayed responses, oscillatory behavior, persistence characteristics, relaxation behavior, hysteresis effects, recovery behavior, or kinetic properties associated with analyte interactions. Such temporal analysis may improve differentiation between true analyte-associated responses and environmentally induced artifacts or noise-induced signal abnormalities.

In certain embodiments, the processing system includes one or more environmental compensation systems configured to compensate for temperature variation, humidity variation, pressure variation, electromagnetic interference, vibration, sample variability, reagent variability, or power supply variability. The environmental compensation systems may receive data from temperature sensors, humidity sensors, inertial sensors, pressure sensors, electromagnetic interference sensors, or environmental monitoring circuitry associated with the sensing platform.

The processing system may further include cloud communication systems configured to transmit acquired sensor data, feature vectors, classification outputs, confidence scores, calibration data, cartridge identifiers, assay metadata, or diagnostic information to remote computing systems. The remote computing systems may perform additional machine learning inference operations, epidemiological analysis operations, mutation tracking operations, assay optimization operations, quality assurance operations, software update operations, or distributed analytics operations.

In certain embodiments, the processing system 700 includes one or more security and authentication systems configured to authenticate cartridges, validate software integrity, encrypt transmitted data, verify sensor authenticity, prevent unauthorized manipulation of assay results, or protect cloud communications. Such security systems may include cryptographic authentication systems, secure enclave processors, public key infrastructure systems, secure boot architectures, blockchain verification systems, authentication tokens, or encrypted communication protocols.

The processing system may further include one or more output generation systems configured to generate analyte determinations, mutation identifications, confidence reports, waveform visualizations, trend analyses, epidemiological alerts, quality control reports, calibration notifications, or machine-readable result outputs. The generated outputs may be displayed locally on a reader instrument, transmitted to a mobile device, uploaded to a cloud platform, integrated into electronic medical record systems, transmitted to laboratory information systems, or stored within distributed databases.

Alternative embodiments associated with FIG. 7 may utilize centralized cloud-processing architectures, edge-computing architectures, distributed inference systems, FPGA-accelerated classification systems, GPU-accelerated machine learning systems, ASIC-based signal processing engines, mobile-device-based classification systems, or combinations thereof. In certain embodiments, the signal processing operation includes aggregation of classifications or signal characteristics from multiple sensing regions associated with the same analyte target before generation of a final analyte determination.

Certain embodiments may utilize commercially available machine learning frameworks, analog front-end integrated circuits, cloud computing infrastructures, signal processing libraries, neural network accelerators, statistical analysis systems, or distributed database systems adapted to perform the sensor processing and classification operations described herein.

In alternative embodiments, the processing system forms part of an integrated analyte detection architecture configured to transform electrical responses generated across a plurality of sensing regions into analyte determinations based on signal preprocessing 704, feature extraction, machine-learning classification, sensor aggregation, confidence scoring 724, temporal analysis, and distributed computational processing operations. The interaction between the feature extraction engine 736, classification engines 738, model databases, classification outputs, aggregation systems, confidence-scoring engine, cloud communication systems, and associated sensing platforms enables robust system-level analyte detection using aggregated electrical responses generated by a distributed sensor array.

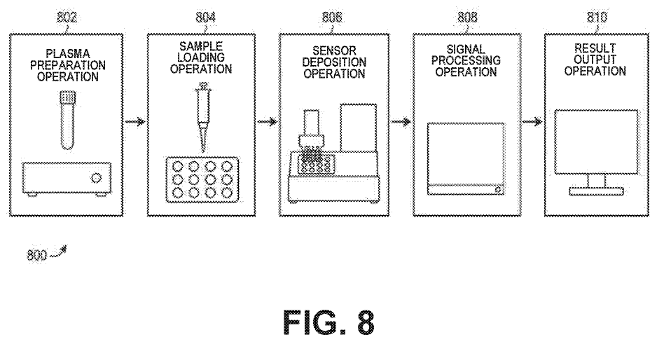

FIG. 8 illustrates an example workflow 800 for performing a detection assay. The workflow 800 includes a sequence of operations configured to prepare a sample, introduce the sample into a sensing cartridge, distribute the sample across a plurality of sensing regions, acquire electrical response data associated with analyte interactions, process the acquired sensor data, classify electrical response characteristics, aggregate classifications across multiple sensing regions, and generate one or more assay outputs associated with analyte detection. In certain embodiments, the workflow 800 is implemented using a combination of cartridge assemblies, sensor arrays, reader instruments, fluidic systems, analog front-end circuitry, local processing systems, cloud computing systems, mobile applications, machine learning systems, or combinations thereof. The workflow 800 may be configured for the detection of nucleic acids, oligonucleotides, proteins, biomarkers, pathogens, mutations, metabolites, toxins, contaminants, chemical compounds, pharmaceutical compounds, or other target analytes.

In alternative embodiments, the workflow 800 begins with a plasma preparation operation 802. The plasma preparation operation 802 may include separation, conditioning, concentration, dilution, purification, stabilization, or preprocessing 704 of a biological sample before introduction into the cartridge assembly. By way of example, the plasma preparation operation 802 may include separation of plasma from whole blood using centrifugation systems, membrane filtration systems, capillary separation systems, microfluidic separation systems, acoustic separation systems, electrophoretic separation systems, density-gradient systems, magnetic separation systems, or combinations thereof.

In alternative embodiments, the plasma preparation operation 802 includes removal of cellular material, particulates, interfering biomolecules, coagulation products, environmental contaminants, or non-target substances that may influence sensing operations. Certain embodiments may include lysis operations, buffering operations, reagent mixing operations, amplification preparation operations, denaturation operations, extraction operations, enzymatic treatment operations, or stabilization operations configured to improve analyte accessibility or signal quality. The plasma preparation operation 802 may further include the introduction of conductive agents, redox agents, amplification reagents, hybridization reagents, blocking agents, enzymes, salts, calibration materials, nanoparticles, or chemically active materials configured to facilitate analyte detection.

The plasma preparation operation 802 may be performed externally using laboratory instrumentation or internally using structures integrated within the cartridge assembly. In certain embodiments, the cartridge assembly includes integrated plasma separation membranes, porous filtration layers, reagent chambers, reaction reservoirs, mixing structures, heating regions, cooling regions, or capillary transport structures configured to perform portions of the plasma preparation operation automatically after sample introduction.

Following plasma preparation, the workflow 800 includes a sample loading operation 804. During the sample loading operation 804, the prepared sample is introduced into a cartridge assembly or sensing platform configured to receive and distribute the sample across a plurality of sensing regions. The sample loading operation may be performed manually using pipettes, syringes, droppers, capillary tubes, transfer devices, or swab applicators. Alternative embodiments may utilize robotic liquid handling systems, automated injection systems, pneumatic delivery systems, electromechanical dispensers, centrifugal loading systems, or integrated microfluidic pumping systems configured to transfer the sample into the cartridge assembly.

In certain embodiments, the sample loading operation 804 includes introduction of the sample through one or more sample ports, reservoirs, injection interfaces, capillary loading regions, puncturable septa, microfluidic channels, loading wells, or fluidic manifolds associated with the cartridge assembly. The cartridge assembly may include venting structures, overflow regions, metering structures, absorbent structures, hydrophilic coatings, hydrophobic barriers, capillary stop structures, or fluid-balancing systems configured to regulate sample movement during loading operations.

The sample loading operation 804 may further include verification operations configured to confirm the successful loading of the sample into the cartridge assembly. Such verification operations may include optical sensing systems, capacitive sensing systems, fluid-level detection systems, pressure sensing systems, impedance monitoring systems, imaging systems, or conductivity measurement systems configured to detect a sample presence, sample volume, fluid continuity, bubble formation, or improper loading conditions. Certain embodiments may generate user notifications or automated error conditions in response to incomplete loading, excessive loading, insufficient sample volume, or air-gap formation within the fluidic pathways.

Following sample loading, the workflow 800 includes a sensor deposition operation 806. During the sensor deposition operation 806, portions of the sample are distributed across multiple sensing regions associated with a sensor sheet or sensing array. The sensor deposition operation may include passive fluid distribution operations, active fluid transport operations, capillary distribution operations, pressure-driven transport operations, centrifugal distribution operations, electroosmotic transport operations, electrowetting operations, pneumatic transport operations, piezoelectric pumping operations, or combinations thereof.

In certain embodiments, the sample is distributed across multiple sensing regions substantially simultaneously through branching microfluidic channels, porous distribution structures, capillary networks, diffusion structures, hydrophilic transport pathways, or fluidic manifolds. Alternative embodiments may sequentially expose subsets of sensing regions to portions of the sample according to predefined timing sequences, analyte-specific assay conditions, amplification schedules, or calibration operations.

The sensor deposition operation 806 may further include analyte interaction processes in which one or more target analytes interact with active sensing surfaces associated with the sensing regions. Such sensing surfaces may include antibodies, nucleic acid probes, aptamers, receptors, conductive polymers, nanostructured coatings, enzymes, molecularly imprinted polymers, graphene structures, metallic nanostructures, hydrogels, or chemically functionalized surfaces configured to selectively interact with target analytes. The analyte interaction processes may include hybridization reactions, electronic reactions, electrochemical reactions, catalytic reactions, redox reactions, charge transfer events, impedance changes, molecular adsorption events, conformational changes, or binding interactions associated with analyte presence.

In certain embodiments, the sensor deposition operation 806 includes environmental control operations configured to maintain operational conditions associated with analyte detection. Such environmental control operations may include ionic regulation, pH regulation, temperature regulation, humidity regulation, thermal cycling, electromagnetic shielding, vibration isolation, atmospheric control, pressure regulation, reagent hydration control, or airflow management operations. Temperature regulation may be performed using resistive heating elements, thermoelectric devices, infrared heating systems, thermal spreaders, phase-change materials, heat sinks, or feedback-controlled thermal management circuitry.

Following analyte interaction and sensor exposure, the workflow 800 includes a signal processing operation 808. During the signal processing operation 808, electrical responses generated by the sensing regions are acquired, digitized, preprocessed, analyzed, classified, aggregated, and interpreted using one or more processing systems. The signal processing operation may include acquisition of electrical current measurements, voltage measurements, impedance measurements, capacitance measurements, resistance measurements, transient waveforms, pulse responses, electrochemical response curves, or frequency-domain measurements generated by the sensing regions. In certain embodiments, analyte determination is performed using aggregated sensor behavior from a subset of sensing regions rather than based exclusively on an output from any individual sensing region.

In certain embodiments, the signal processing operation 808 utilizes analog front-end circuitry including transimpedance amplifiers, instrumentation amplifiers, analog multiplexers, waveform generators, analog-to-digital converters, digital signal processors, excitation signal generators, impedance analyzers, filtering systems, or current sensing circuitry configured to interrogate the sensing regions and acquire electrical responses over time.

The signal processing operation 808 may further include preprocessing 704 operations, including baseline normalization, drift compensation, noise filtering, smoothing operations, transient rejection operations, derivative calculations, interpolation operations, frequency-domain analysis, spectral analysis, adaptive thresholding operations, feature extraction operations, or signal averaging operations configured to improve classification robustness and compensate for environmental variability.

In alternative embodiments, the signal processing operation 808 includes one or more feature extraction engines 736 of FIG. 7 configured to derive measurable signal characteristics associated with the acquired sensor responses. The extracted features may include slope values, derivative values, transient durations, peak amplitudes, waveform shapes, entropy measurements, spectral features, temporal correlation values, rise times, settling times, oscillatory characteristics, impedance characteristics, relaxation properties, phase shifts, signal persistence values, or multidimensional feature vectors associated with the sensor outputs.

Following feature extraction, the signal processing operation 808 may include one or more classification operations performed using rules-based systems, statistical classifiers, neural networks, support vector machines, Bayesian classifiers, random forest models, convolutional neural networks, recurrent neural networks, transformer architectures, ensemble classifiers, fuzzy logic systems, or combinations thereof. The classification systems may classify signals 708 of FIG. 7 as strong positive signals, weak positive signals, strong negative signals, weak negative signals, invalid signals, noisy signals, unstable signals, saturated signals, or inconclusive signals.

In certain embodiments, the signal processing operation 808 includes aggregation operations configured to aggregate classifications or signal characteristics across subsets of sensing regions assigned to common analyte targets. The aggregation operations may include weighted voting systems, confidence scoring 724 of FIG. 7 systems, probabilistic fusion systems, consensus analysis systems, Bayesian inference systems, machine learning aggregation systems, temporal correlation systems, or neural network inference systems configured to generate system-level analyte determinations based on combined sensor responses.

The signal processing operation 808 may further include one or more confidence-scoring engines configured to generate confidence values associated with analyte determinations. Confidence values may be based on the number of responsive sensing regions, signal magnitude consistency, waveform similarity, temporal agreement, calibration parameters, environmental compensation factors, historical sensor reliability, machine-learning confidence outputs, or combinations thereof. In certain embodiments, analyte presence is determined when one or more strong positive classifications are detected within a subset of sensing regions or when multiple weak positive classifications collectively exceed one or more confidence thresholds.

Following completion of the signal processing operation 808, the workflow 800 includes a result output operation 810. During the result output operation 810, one or more assay outputs are generated and communicated to a user, remote system, database, or external computing platform. The assay outputs may include positive or negative analyte determinations, mutation detection outputs, quantitative concentration values, confidence scores, waveform visualizations, temporal response graphs, epidemiological alerts, quality control indicators, calibration notifications, assay status indicators, or historical comparison data.

The result output operation 810 may include display of assay results on a local reader instrument, transmission of assay results to a mobile application, upload of assay results to a cloud computing platform, generation of downloadable reports, integration with electronic medical record systems, transmission to laboratory information systems, or communication with epidemiological monitoring platforms. In certain embodiments, the result output operation 810 includes encrypted transmission of assay data using secure communication protocols, authentication systems, secure cloud infrastructures, blockchain verification systems, or secure enclave processors.

The workflow 800 may further include quality assurance operations configured to evaluate assay integrity, cartridge validity, calibration status, environmental stability, reagent condition, sensor performance, or data integrity. Such quality assurance operations may include evaluation of reference sensing regions, calibration sensing regions, environmental sensors, control reagents, timing validation systems, or cloud-based verification systems. In alternative embodiments, invalid or inconclusive assay outputs are generated in response to detected abnormalities, control failures, environmental instability, or signal integrity deficiencies.

Alternative embodiments associated with the workflow 800 may utilize decentralized point-of-care workflows, centralized laboratory workflows, field-deployable workflows, continuous monitoring workflows, wearable sensing workflows, automated robotic workflows, cloud-native analytics workflows, edge-computing workflows, or distributed sensing network workflows. Certain embodiments may utilize commercially available liquid-handling systems, electrochemical analyzers, analog front-end integrated circuits, cloud computing infrastructures, machine-learning software frameworks, microfluidic systems, thermal control systems, or wireless communication systems adapted to perform the operations described herein.

In alternative embodiments, the workflow 800 forms part of an integrated analyte detection architecture configured to transform physical and chemical interactions occurring within a plurality of sensing regions into computationally derived analyte determinations based on distributed sensor responses, signal classification, aggregation analysis, confidence scoring 724 of FIG. 7, machine learning inference, and cloud-based analytics operations. The interaction between the plasma preparation operation 802, sample loading operation 804, sensor deposition operation 806, signal processing operation 808, and result output operation 810 enables robust analyte detection using redundant sensor architectures and system-level signal interpretation across a distributed sensor array.

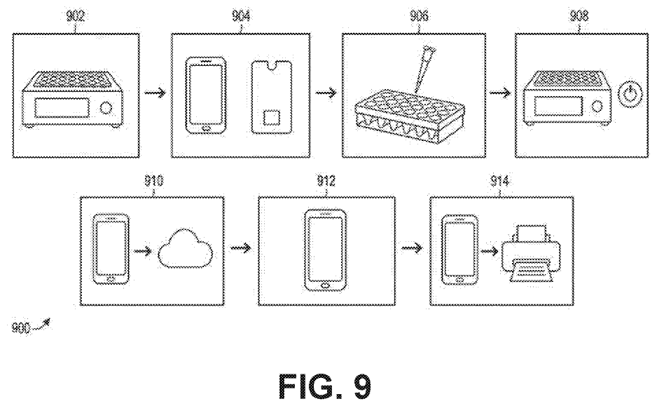

FIG. 9 illustrates an operational sequence 900 for performing a test using the system. The operational sequence 900 includes a sequence of user interactions, device operations, cartridge operations, signal acquisition operations, computational processing operations, and output generation operations associated with performing an analyte detection assay using the disclosed sensing system. In certain embodiments, the operational sequence 900 is implemented using a reader instrument, a cartridge assembly including a plurality of sensing regions, one or more processing systems, mobile computing devices, cloud computing infrastructures, communication systems, or combinations thereof. The operational sequence 900 may be utilized in centralized laboratory environments, decentralized point-of-care environments, field testing environments, industrial monitoring environments, research laboratories, healthcare facilities, mobile diagnostic environments, at-home testing environments, or distributed sensing networks.

In alternative embodiments, the operational sequence 900 begins with a reader setup operation 902. During the reader setup operation 902, a reader instrument or processing device is initialized, powered, authenticated, calibrated, configured, or otherwise prepared for operation. The reader instrument may include one or more processors, analog front-end circuits, communication modules, displays, user interfaces, power systems, environmental monitoring systems, or signal acquisition systems configured to interface with a cartridge assembly containing a plurality of sensors.

The reader setup operation 902 may include activation of one or more power systems, including rechargeable battery systems, external power adapters, USB power interfaces, wireless charging systems, or internal voltage regulation systems. In certain embodiments, the reader setup operation includes execution of initialization firmware, boot routines, operating system instructions, self-diagnostic operations, memory verification operations, communication interface initialization operations, environmental sensor initialization operations, or analog front-end calibration operations.

The reader setup operation 902 may further include verification of operational readiness associated with one or more subsystems of the reader instrument. Such subsystems may include analog signal acquisition circuitry, impedance measurement systems, current sensing circuitry, temperature regulation systems, wireless communication systems, displays, touchscreen interfaces, storage systems, security systems, or cloud communication systems. In alternative embodiments, the reader instrument performs internal self-check operations configured to identify calibration drift, signal acquisition abnormalities, memory corruption, hardware failures, environmental instability, communication failures, or power management abnormalities before initiation of a test sequence.

In certain embodiments, the reader setup operation 902 includes the establishment of communication between the reader instrument and one or more external computing systems. The external computing systems may include smartphones, tablets, cloud computing platforms, remote databases, laboratory information systems, electronic medical record systems, epidemiological monitoring systems, or distributed analytics systems. Communication may be established using Bluetooth communication systems, Wi-Fi communication systems, cellular communication systems, NFC systems, Ethernet systems, USB communication systems, or other wired or wireless communication architectures.

Following reader setup, the operational sequence 900 includes a cartridge registration operation 904. During the cartridge registration operation 904, a cartridge assembly configured for analyte detection is identified, authenticated, associated with assay parameters, or linked to one or more software systems. In defined embodiments, the cartridge assembly includes one or more identification structures such as QR codes, barcodes, RFID tags, NFC devices, EEPROM memory devices, authentication integrated circuits, optical encoding structures, magnetic encoding systems, or physically unclonable function structures configured to store cartridge-related information.

The cartridge registration operation 904 may include acquisition of cartridge identification information using imaging systems, barcode scanners, RFID readers, optical sensors, wireless communication interfaces, or electrical contact interfaces associated with the reader instrument or mobile computing device. The acquired cartridge identification information may include assay identifiers, analyte panels, manufacturing data, expiration information, calibration parameters, lot numbers, reagent information, firmware compatibility information, cloud authentication data, or machine learning model references associated with the cartridge assembly.

In certain embodiments, the cartridge registration operation 904 includes transmission of cartridge identification information to one or more cloud computing systems configured to validate cartridge authenticity, retrieve assay-specific processing parameters, retrieve calibration information, retrieve machine learning models, verify expiration status, verify software compatibility, or associate the cartridge with a user account or testing session. Alternative embodiments may include cryptographic authentication operations, secure cloud validation operations, blockchain verification systems, token-based authentication systems, or encrypted communication systems configured to reduce unauthorized cartridge usage or counterfeit cartridge deployment.

Following cartridge registration, the operational sequence 900 includes a sample loading operation 906. During the sample loading operation 906, a sample is introduced into the cartridge assembly for analysis. The sample may comprise blood, plasma, serum, saliva, urine, tissue extracts, swab extracts, environmental samples, industrial samples, agricultural samples, food extracts, chemical solutions, or other analyte-containing materials. The sample loading operation may be performed manually using pipettes, syringes, droppers, swabs, capillary devices, or transfer tools. Alternative embodiments may utilize robotic liquid handling systems, automated injection systems, pneumatic transport systems, electromechanical dispensing systems, or integrated microfluidic loading architectures.

In certain embodiments, the sample loading operation 906 includes introduction of the sample through one or more sample ports, reservoirs, loading wells, capillary channels, puncturable membranes, injection interfaces, or microfluidic manifolds associated with the cartridge assembly. The cartridge assembly may include fluidic distribution structures configured to transport portions of the sample to multiple sensing regions distributed throughout a sensor array. Such fluidic distribution structures may include capillary channels, porous membranes, branching manifolds, hydrophilic transport pathways, diffusion structures, electrowetting systems, centrifugal transport systems, or pressure-driven flow systems.

The sample loading operation 906 may further include verification operations configured to confirm successful sample introduction into the cartridge assembly. Verification operations may utilize optical sensing systems, impedance sensing systems, fluid-level detection systems, conductivity sensing systems, pressure sensing systems, imaging systems, capacitive sensing systems, or flow monitoring systems configured to detect sample presence, sample continuity, fluid distribution, bubble formation, insufficient loading conditions, or overflow conditions.

Following sample loading, the operational sequence 900 includes a test initiation operation 908. During the test initiation operation 908, the reader instrument or associated processing system initiates one or more assay sequences configured to interrogate the sensing regions of the cartridge assembly. In alternative embodiments, the test initiation operation includes activation of analog front-end circuitry, generation of excitation waveforms, initialization of timing sequences, activation of thermal control systems, enabling of multiplexing systems, synchronization of signal acquisition systems, or initiation of cloud communication operations.

The test initiation operation 908 may include application of electrical bias conditions, alternating current excitation signals, pulse waveforms, impedance spectroscopy sequences, voltage sweeps, current injection sequences, frequency-domain interrogation signals, or programmable waveform patterns to sensing regions distributed throughout the sensor array. Such interrogation signals may be generated using waveform generators, digital-to-analog converters, analog multiplexers, impedance analyzers, current drivers, or programmable signal generation systems associated with the reader instrument.

In certain embodiments, the test initiation operation 908 includes environmental control operations configured to regulate operational conditions associated with the assay. Such environmental control operations may include temperature regulation, thermal cycling, humidity regulation, vibration isolation, electromagnetic shielding, airflow management, pressure regulation, or reagent hydration control. Temperature regulation may be performed using resistive heating elements, thermoelectric devices, thermal spreaders, infrared heating systems, heat sinks, or closed-loop thermal feedback systems.

Following initiation of the test, the operational sequence 900 includes a data transmission operation 910. During the data transmission operation 910, sensor data acquired from the sensing regions is transmitted to one or more processing systems configured to perform signal processing, classification, aggregation, and analyte determination operations. The data transmission operation may include transmission of raw electrical signals, processed feature vectors, waveform data, calibration information, environmental sensor data, cartridge identifiers, timestamp information, or metadata associated with the assay operation. In certain embodiments, transmitted data includes classifications, extracted signal characteristics, waveform parameters, temporal response characteristics, feature vectors, confidence scores, or aggregated sensor outputs associated with subsets of sensors assigned to the same analyte target.

In alternative embodiments, the data transmission operation 910 utilizes wired communication systems, wireless communication systems, Bluetooth systems, Wi-Fi systems, cellular communication systems, NFC systems, Ethernet systems, serial communication interfaces, cloud communication infrastructures, or combinations thereof. Sensor data may be transmitted continuously, periodically, asynchronously, synchronously, or according to predefined transmission intervals associated with the assay protocol.

The data transmission operation 910 may further include encryption operations, compression operations, authentication operations, packet verification operations, error correction operations, buffering operations, or synchronization operations configured to improve communication reliability and protect transmitted assay data. Alternative embodiments may utilize secure communication protocols, public key infrastructure systems, secure enclave processors, token-based authentication systems, blockchain verification systems, or cloud-based validation systems configured to protect assay integrity and user privacy.

Following transmission of sensor data, the operational sequence 900 includes a result viewing operation 912. During the result viewing operation 912, one or more analyte detection results or assay outputs are generated and presented to a user, a remote system, a cloud platform, or an external database. The result viewing operation may include display of positive or negative analyte determinations, mutation detection outputs, concentration measurements, confidence scores, waveform visualizations, temporal response graphs, epidemiological alerts, assay quality indicators, calibration notifications, environmental status indicators, or historical comparison information.

In alternative embodiments, the results are displayed using a display integrated within the reader instrument. Alternative embodiments may present results through mobile applications, cloud dashboards, remote monitoring platforms, electronic medical record systems, laboratory information systems, printed reports, wearable devices, augmented reality systems, or voice-based interfaces. The displayed results may include graphical visualizations, numerical values, color-coded indicators, probability values, trend graphs, multidimensional plots, or machine-readable outputs configured for downstream analysis.

The result viewing operation 912 may further include user interaction operations configured to permit storage, export, annotation, transmission, comparison, or interpretation of assay results. In certain embodiments, users may electronically transmit results to healthcare providers, laboratory systems, cloud databases, epidemiological monitoring systems, industrial monitoring platforms, or regulatory systems using integrated communication interfaces associated with the sensing platform.

Following result viewing, the operational sequence 900 includes a result output operation 914. During the result output operation 914, assay results are finalized, stored, archived, transmitted, printed, or integrated into one or more downstream systems. The result output operation may include the generation of structured data records, cloud storage operations, audit logging operations, historical database updates, machine learning training data generation, epidemiological aggregation operations, regulatory reporting operations, or quality assurance documentation operations.

In defined embodiments, the result output operation 914 includes transmission of assay outputs to cloud computing systems configured to perform epidemiological monitoring, mutation tracking, distributed analytics, machine learning retraining, calibration optimization, assay quality analysis, or longitudinal population monitoring. The transmitted outputs may include anonymized assay data, waveform data, classification results 708, confidence scores, environmental data, geographic data, timestamp information, or cartridge metadata associated with the assay session.

The operational sequence 900 may further include post-test operations configured to clean, disinfect, calibrate, power down, update, or otherwise maintain the reader instrument following completion of the assay. Such post-test operations may include cartridge removal operations, cartridge disposal operations, automated cleaning sequences, ultraviolet sterilization operations, firmware update operations, battery charging operations, cloud synchronization operations, memory management operations, or recalibration operations associated with the sensing platform.

Alternative embodiments associated with the operational sequence 900 may utilize automated laboratory workflows, portable field-testing workflows, wearable sensing workflows, continuous monitoring workflows, decentralized point-of-care workflows, robotic testing systems, cloud-native analytics workflows, or distributed sensor network architectures. Certain embodiments may utilize commercially available mobile computing devices, cloud computing infrastructures, analog front-end integrated circuits, wireless communication systems, machine learning software frameworks, electrochemical analyzers, microfluidic systems, or secure authentication systems adapted to perform the operational functions described herein.

In certain embodiments, the operational sequence 900 forms part of an integrated analyte detection architecture configured to coordinate cartridge registration, sample handling, sensor interrogation, signal acquisition, computational processing, cloud communication, result generation, and downstream analytics operations associated with distributed sensor-based analyte detection. The interaction between the reader setup operation 902, cartridge registration operation 904, sample loading operation 906, test initiation operation 908, data transmission operation 910, result viewing operation 912, and result output operation 914 enables robust analyte detection using redundant sensing architectures, aggregated sensor analysis, machine learning classification systems, and cloud-connected processing infrastructures.

In defined embodiments, analyte determination may be performed using aggregated classifications, aggregated signal characteristics, aggregated feature vectors, aggregated waveform characteristics, aggregated temporal response characteristics, aggregated spectral characteristics, or combinations thereof derived from multiple sensing regions associated with the same analyte target. The disclosed systems may evaluate current characteristics, voltage characteristics, impedance characteristics, capacitance characteristics, waveform characteristics, frequency-domain characteristics, phase characteristics, conductivity characteristics, electrochemical characteristics, ionic strength measurements, ionic concentration measurements, ionic activity measurements, pH measurements or combinations thereof over time. In certain embodiments, analyte determination is performed using combined sensor behavior rather than based exclusively on any individual sensor response, thereby improving robustness, fault tolerance, reproducibility, and resistance to sensor variability.

The foregoing has described the principles, embodiments, and modes of operation of certain embodiments. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of certain embodiments as defined by the following claims.

What is claimed is:

1. A system for detecting an analyte, comprising:
a cartridge comprising a plurality of electrically addressable sensors arranged in an array, wherein a subset of the plurality of electrically addressable sensors is associated with a same analyte target;
a signal acquisition device coupled to the cartridge and configured to acquire electrical signals from the plurality of electrically addressable sensors;
a signal processing device coupled to the signal acquisition device and configured to preprocess and classify the electrical signals based on one or more electrical characteristics associated with the electrical signals over time;
an aggregation processor coupled to the signal processing device and configured to aggregate classifications and signal characteristics from the subset of the plurality of electrically addressable sensors associated with the same analyte target;
wherein the aggregation processor is configured to determine a presence of the analyte when multiple non-definitive classifications from the subset of the plurality of electrically addressable sensors collectively satisfy a confidence threshold in the absence of a definitive positive classification;
a confidence scoring processor coupled to the aggregation processor and configured to determine at least one of a presence or absence of the analyte based on the aggregated classifications and signal characteristics; and
wherein the signal processing device is further configured to classify one or more invalid electrical signals if at least one of temporal instability, substantially flat signal behavior, or excessive signal noise is detected, and to exclude the invalid electrical signals from analyte determination.

2. The system of claim 1, wherein the one or more electrical characteristics comprise at least one of electrical current, voltage, impedance, capacitance, resistance, waveform shape, frequency-domain characteristics, transient response characteristics, phase characteristics, binding kinetics, ionic strength measurements, ionic concentration measurements, ionic activity measurements, pH measurements, electrochemical response characteristics or combinations thereof, evaluated over time.

3. The system of claim 1, wherein the signal processing device is further configured to preprocess the electrical signals using at least one of filtering, baseline normalization, drift compensation, smoothing, transient rejection, adaptive thresholding, derivative analysis, interpolation, or spectral analysis.

4. The system of claim 1, wherein the aggregation processor is further configured to aggregate classifications from the subset of the plurality of electrically addressable sensors based on temporal correlation between electrical signals generated by the subset of the plurality of electrically addressable sensors.

5. The system of claim 1, wherein the confidence scoring processor is further configured to generate a confidence score based on at least one of a quantity of sensors classified as positive, signal magnitude characteristics, signal consistency across the subset of the plurality of electrically addressable sensors, or waveform similarity characteristics.

6. The system of claim 1, wherein the signal processing device is further configured to classify the electrical signals.

7. The system of claim 1, wherein the cartridge further comprises one or more fluid distribution structures configured to distribute a sample across the subset of the plurality of electrically addressable sensors associated with the same analyte target.

8. A system for detecting an analyte, comprising:
a cartridge comprising a plurality of electrically addressable sensors arranged in an array, wherein a subset of the plurality of electrically addressable sensors is associated with a same analyte target;
a signal acquisition device coupled to the cartridge and configured to acquire electrical signals from the plurality of electrically addressable sensors;
wherein the signal acquisition device is further configured to apply one or more excitation conditions to the plurality of electrically addressable sensors and acquire corresponding electrical responses over time;
a signal processing device coupled to the signal acquisition device and configured to preprocess and classify the electrical signals based on one or more electrical characteristics associated with the electrical signals over time;
an aggregation processor coupled to the signal processing device and configured to aggregate classifications and signal characteristics from the subset of the plurality of electrically addressable sensors associated with the same analyte target;
wherein the aggregation processor is configured to determine a presence of the analyte when multiple non-definitive classifications from the subset of the plurality of electrically addressable sensors collectively satisfy a confidence threshold in the absence of a definitive positive classification;
a confidence scoring processor coupled to the aggregation processor and configured to determine at least one of a presence or absence of the analyte based on the aggregated classifications and signal characteristics; and
wherein the signal processing device is further configured to classify one or more invalid electrical signals if at least one of temporal instability, substantially flat signal behavior, or excessive signal noise is detected, and to exclude the invalid electrical signals from analyte determination.

9. The system of claim 8, wherein the one or more electrical characteristics comprise at least one of electrical current, voltage, impedance, capacitance, resistance, waveform shape, frequency-domain characteristics, transient response characteristics, phase characteristics, binding kinetics, ionic strength measurements, ionic concentration measurements, ionic activity measurements, pH measurements, electrochemical response characteristics or combinations thereof, evaluated over time.

10. The system of claim 8, wherein the signal processing device is further configured to preprocess the electrical signals using at least one of filtering, baseline normalization, drift compensation, smoothing, transient rejection, adaptive thresholding, derivative analysis, interpolation, or spectral analysis.

11. The system of claim 8, wherein the aggregation processor is further configured to aggregate classifications from the subset of the plurality of electrically addressable sensors based on temporal correlation between electrical signals generated by the subset of the plurality of electrically addressable sensors.

12. The system of claim 8, wherein the confidence scoring processor is further configured to generate a confidence score based on at least one of a quantity of sensors classified as positive, signal magnitude characteristics, signal consistency across the subset of the plurality of electrically addressable sensors, or waveform similarity characteristics.

13. The system of claim 8, wherein the signal processing device is further configured to classify the electrical signals.

14. The system of claim 8, wherein the cartridge further comprises one or more fluid distribution structures configured to distribute a sample across the subset of the plurality of electrically addressable sensors associated with the same analyte target.

15. A system for detecting an analyte, comprising:
a cartridge comprising a plurality of electrically addressable sensors arranged in an array, wherein a subset of the plurality of electrically addressable sensors is associated with a same analyte target;
wherein the cartridge further comprises one or more fluid distribution structures configured to distribute a sample across the subset of the plurality of electrically addressable sensors associated with the same analyte target;
a signal acquisition device coupled to the cartridge and configured to acquire electrical signals from the plurality of electrically addressable sensors;
wherein the signal acquisition device is further configured to apply one or more excitation conditions to the plurality of electrically addressable sensors and acquire corresponding electrical responses over time;
a signal processing device coupled to the signal acquisition device and configured to preprocess and classify the electrical signals based on one or more electrical characteristics associated with the electrical signals over time;
an aggregation processor coupled to the signal processing device and configured to aggregate classifications and signal characteristics from the subset of the plurality of electrically addressable sensors associated with the same analyte target;
wherein the aggregation processor is configured to determine a presence of the analyte when multiple non-definitive classifications from the subset of the plurality of electrically addressable sensors collectively satisfy a confidence threshold in the absence of a definitive positive classification;
a confidence scoring processor coupled to the aggregation processor and configured to determine at least one of a presence or absence of the analyte based on the aggregated classifications and signal characteristics; and
wherein the signal processing device is further configured to classify one or more invalid electrical signals if at least one of temporal instability, substantially flat signal behavior, or excessive signal noise is detected and to exclude the invalid electrical signals from analyte determination.

16. The system of claim 15, wherein the one or more electrical characteristics comprise at least one of electrical current, voltage, impedance, capacitance, resistance, waveform shape, frequency-domain characteristics, transient response characteristics, phase characteristics, binding kinetics, ionic strength measurements, ionic concentration measurements, ionic activity measurements, pH measurements, electrochemical response characteristics or combinations thereof, evaluated over time.

17. The system of claim 15, wherein the signal processing device is further configured to preprocess the electrical signals using at least one of filtering, baseline normalization, drift compensation, smoothing, transient rejection, adaptive thresholding, derivative analysis, interpolation, or spectral analysis.

18. The system of claim 15, wherein the aggregation processor is further configured to aggregate classifications from the subset of the plurality of electrically addressable sensors based on temporal correlation between electrical signals generated by the subset of the plurality of electrically addressable sensors.

19. The system of claim 15, wherein the confidence scoring processor is further configured to generate a confidence score based on at least one of a quantity of sensors classified as positive, signal magnitude characteristics, signal consistency across the subset of the plurality of electrically addressable sensors, or waveform similarity characteristics.

20. The system of claim 15, wherein the signal processing device is further configured to classify the electrical signals.
